SEQUENCE LISTING (275 sequences)

SEQ ID NO: 1 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
GFTFRDYA

SEQ ID NO: 4 - protein, 8 aa, synthetic construct
ISGSGGNT

SEQ ID NO: 5 - protein, 18 aa, synthetic construct
AKDRLSITIRPRYYGLDV

SEQ ID NO: 6 - protein, 11 aa, synthetic construct
QSLLYSIGYNY

SEQ ID NO: 7 - skipped

SEQ ID NO: 8 - protein, 9 aa, synthetic construct
MQALQTPYT

SEQ ID NO: 9 - protein, 452 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, synthetic construct
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, synthetic construct
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, synthetic construct
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, synthetic construct
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, synthetic construct
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, synthetic construct
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, synthetic construct
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, synthetic construct
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, synthetic construct
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, synthetic construct
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, synthetic construct
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, synthetic construct
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, synthetic construct
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, synthetic construct
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, synthetic construct
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, synthetic construct
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, synthetic construct; identical to SEQ ID NO: 69

SEQ ID NO: 74 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, synthetic construct; identical to SEQ ID NO: 74

SEQ ID NO: 77 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, synthetic construct; identical to SEQ ID NO: 74

SEQ ID NO: 81 - protein, 116 aa, synthetic construct; identical to SEQ ID NO: 79

SEQ ID NO: 82 - protein, 111 aa, synthetic construct; identical to SEQ ID NO: 78

SEQ ID NO: 83 - protein, 116 aa, synthetic construct; identical to SEQ ID NO: 69

SEQ ID NO: 84 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, synthetic construct; identical to SEQ ID NO: 75

SEQ ID NO: 86 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, synthetic construct; identical to SEQ ID NO: 87

SEQ ID NO: 90 - protein, 111 aa, synthetic construct; identical to SEQ ID NO: 84

SEQ ID NO: 91 - protein, 116 aa, synthetic construct; identical to SEQ ID NO: 75

SEQ ID NO: 92 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, synthetic construct; identical to SEQ ID NO: 69

SEQ ID NO: 94 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, synthetic construct; identical to SEQ ID NO: 69

SEQ ID NO: 96 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, synthetic construct; identical to SEQ ID NO: 69

SEQ ID NO: 98 - protein, 111 aa, synthetic construct; identical to SEQ ID NO: 78

SEQ ID NO: 99 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, synthetic construct; identical to SEQ ID NO: 99

SEQ ID NO: 102 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, synthetic construct; identical to SEQ ID NO: 99

SEQ ID NO: 104 - protein, 111 aa, synthetic construct; identical to SEQ ID NO: 78

SEQ ID NO: 105 - protein, 116 aa, synthetic construct; identical to SEQ ID NO: 77

SEQ ID NO: 106 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, synthetic construct; identical to SEQ ID NO: 77

SEQ ID NO: 110 - protein, 111 aa, synthetic construct
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, synthetic construct
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, synthetic construct
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, synthetic construct
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, synthetic construct; identical to SEQ ID NO: 77

SEQ ID NO: 118 - protein, 111 aa, synthetic construct; identical to SEQ ID NO: 114

SEQ ID NO: 119 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, synthetic construct
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, synthetic construct
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, synthetic construct
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, synthetic construct; identical to SEQ ID NO: 77

SEQ ID NO: 126 - protein, 111 aa, synthetic construct
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, synthetic construct; identical to SEQ ID NO: 77

SEQ ID NO: 128 - protein, 111 aa, synthetic construct
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, synthetic construct; identical to SEQ ID NO: 77

SEQ ID NO: 130 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, synthetic construct
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, synthetic construct
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, synthetic construct
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, synthetic construct; identical to SEQ ID NO: 114

SEQ ID NO: 135 - protein, 116 aa, synthetic construct; identical to SEQ ID NO: 77

SEQ ID NO: 136 - protein, 111 aa, synthetic construct; identical to SEQ ID NO: 124

SEQ ID NO: 137 - protein, 116 aa, synthetic construct; identical to SEQ ID NO: 107

SEQ ID NO: 138 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, synthetic construct
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, synthetic construct
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, synthetic construct; identical to SEQ ID NO: 107

SEQ ID NO: 142 - protein, 111 aa, synthetic construct; identical to SEQ ID NO: 114

SEQ ID NO: 143 - protein, 116 aa, synthetic construct; identical to SEQ ID NO: 113

SEQ ID NO: 144 - protein, 111 aa, synthetic construct
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, synthetic construct; identical to SEQ ID NO: 77

SEQ ID NO: 146 - protein, 111 aa, synthetic construct; identical to SEQ ID NO: 138

SEQ ID NO: 147 - protein, 116 aa, synthetic construct
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, synthetic construct
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, synthetic construct
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, synthetic construct
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, synthetic construct
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, synthetic construct
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, synthetic construct
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, synthetic construct
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, synthetic construct
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, synthetic construct
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, synthetic construct
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, synthetic construct
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, synthetic construct
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, synthetic construct
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, synthetic construct
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, synthetic construct
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, synthetic construct
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, synthetic construct
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, synthetic construct
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, synthetic construct
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, synthetic construct
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, synthetic construct
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, synthetic construct
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, synthetic construct
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, synthetic construct
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, synthetic construct
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, synthetic construct
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, synthetic construct
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, synthetic construct
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, synthetic construct
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, synthetic construct; identical to SEQ ID NO: 177

SEQ ID NO: 179 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, synthetic construct
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, synthetic construct; identical to SEQ ID NO: 177

SEQ ID NO: 182 - protein, 111 aa, synthetic construct
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, synthetic construct
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, synthetic construct
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, synthetic construct
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, synthetic construct
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, synthetic construct
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, synthetic construct
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, synthetic construct
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, synthetic construct
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, synthetic construct
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, synthetic construct
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, synthetic construct
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, synthetic construct
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, synthetic construct
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, synthetic construct
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, synthetic construct
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, synthetic construct
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, synthetic construct
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, synthetic construct
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, synthetic construct
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, synthetic construct
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, synthetic construct
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, synthetic construct
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, synthetic construct
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, synthetic construct
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, synthetic construct
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, synthetic construct
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, synthetic construct
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, synthetic construct
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, synthetic construct
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, synthetic construct
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, synthetic construct
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, synthetic construct
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, synthetic construct
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, synthetic construct; identical to SEQ ID NO: 210

SEQ ID NO: 219 - protein, 120 aa, synthetic construct
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, synthetic construct
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, synthetic construct
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, synthetic construct
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, synthetic construct
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, synthetic construct
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, synthetic construct
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, synthetic construct
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, synthetic construct
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, synthetic construct; identical to SEQ ID NO: 227

SEQ ID NO: 229 - protein, 108 aa, synthetic construct
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, synthetic construct; identical to SEQ ID NO: 229

SEQ ID NO: 231 - protein, 108 aa, synthetic construct; identical to SEQ ID NO: 227

SEQ ID NO: 232 - protein, 108 aa, synthetic construct; identical to SEQ ID NO: 229

SEQ ID NO: 233 - protein, 108 aa, synthetic construct
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, synthetic construct
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, synthetic construct
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, synthetic construct
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, synthetic construct
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, synthetic construct
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, synthetic construct
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, synthetic construct; identical to SEQ ID NO: 234

SEQ ID NO: 242 - protein, 108 aa, synthetic construct
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, synthetic construct
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 244 - protein, 118 aa, synthetic construct
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 245 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 246 - protein, 121 aa, synthetic construct
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 247 - protein, 118 aa, synthetic construct
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 248 - protein, 118 aa, synthetic construct
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 249 - protein, 120 aa, synthetic construct
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 250 - protein, 118 aa, synthetic construct
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 251 - protein, 118 aa, synthetic construct
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 252 - protein, 118 aa, synthetic construct
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 253 - protein, 118 aa, synthetic construct
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 254 - protein, 118 aa, synthetic construct
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 255 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 256 - protein, 118 aa, synthetic construct
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 257 - protein, 118 aa, synthetic construct; identical to SEQ ID NO: 254

SEQ ID NO: 258 - protein, 118 aa, synthetic construct
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 259 - protein, 118 aa, synthetic construct
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 260 - protein, 107 aa, synthetic construct
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 261 - protein, 111 aa, synthetic construct
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 262 - protein, 107 aa, synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 263 - protein, 111 aa, synthetic construct
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 264 - protein, 111 aa, synthetic construct
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 265 - protein, 111 aa, synthetic construct; identical to SEQ ID NO: 261

SEQ ID NO: 266 - protein, 111 aa, synthetic construct
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 267 - protein, 111 aa, synthetic construct
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 268 - protein, 107 aa, synthetic construct; identical to SEQ ID NO: 260

SEQ ID NO: 269 - protein, 107 aa, synthetic construct
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 270 - protein, 107 aa, synthetic construct
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 271 - protein, 107 aa, synthetic construct
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 272 - protein, 107 aa, synthetic construct
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 273 - protein, 107 aa, synthetic construct
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 274 - protein, 107 aa, synthetic construct
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 275 - protein, 107 aa, synthetic construct
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

(19) United States
(12) Patent Application Publication
(10) Pub. No.: US 2024/0141051 A1
(43) Pub. Date: May 2, 2024

(54) METHODS FOR TREATING HAND AND FOOT DERMATITIS BY ADMINISTERING AN IL-4R ANTAGONIST

(71) Applicant: REGENERON PHARMACEUTICALS, INC., Tarrytown, NY (US)

(72) Inventors: Ashish BANSAL, White Plains, NY (US); Jingdong CHAO, Tarrytown, NY (US)

(21) Appl. No.: 18/499,679

(22) Filed: Nov. 1, 2023

Related U.S. Application Data
(60) Provisional application No. 63/381,908, filed on Nov. 1, 2022.
(60) Provisional application No. 63/489,377, filed on Mar. 9, 2023.

(51) Int. Cl. C07K 16/28; A61P 17/00

(52) U.S. Cl. CPC C07K 16/2866; A61P 17/00; C07K 2317/21

(57) ABSTRACT

Methods for treating moderate-to-severe atopic dermatitis of the hand and/or foot in a subject are provided. In one aspect, the methods comprise administering to the subject one or more doses of an interleukin-4 receptor (IL-4R) antagonist, such as an anti-IL-4R antibody or antigen-binding fragment thereof.

### REFERENCE TO A SEQUENCE LISTING XML

This application contains a Sequence Listing which has been submitted electronically in XML format. The Sequence Listing XML is incorporated herein by reference. Said XML file, created on Oct. 25, 2023, is named 40848_0117 USU1_SL.xml and is 267,878 bytes in size.

### CROSS-REFERENCE TO RELATED APPLICATIONS

This application claims priority to United States Provisional Patent Application Nos. 63/381,908, filed Nov. 1, 2022, and 63/489,377, filed Mar. 9, 2023, the contents of each of which are incorporated by reference herein.

### FIELD OF THE INVENTION

The present disclosure relates to the use of interleukin-4 receptor (IL-4R) antagonists for treating hand and/or foot dermatitis.

### BACKGROUND

Hand and foot dermatitis is an umbrella term that includes irritant contact dermatitis (ICD), allergic contact dermatitis (ACD) and patients with a history of, or presence of concurrent atopic dermatitis (AD), which is also known as atopic hand and foot dermatitis (Agner, et al., J Eur Acad Dermatol Venereol, 2015, 29:2417-2422). The morphological types that have been described in this condition include the following: vesicular (dyshidrotic), hyperkeratotic, fissured, and nummular (Menne, et al., Contact Dermatitis, 2011, 65:3-12).

Patients with atopic dermatitis (AD) are at increased risk of hand dermatitis of any etiology as compared to the general population with a 3- to 4-fold increase of prevalence as compared to controls (Ruff, et al., Br J Dermatol, 2018, 178:879-888). The prevalence of hand dermatitis in patients with AD has been reported to be approximately 60% (Simpson, et al., Dermatitis, 2006 17:123-127). Similarly, the prevalence of foot dermatitis in patients with AD has been reported to be approximately 30% (Holm, et al., J Eur Acad Dermatol Venereol, 2016, 30:1760-1767). Atopic dermatitis is a known risk factor for development of hand dermatitis with approximately 28% of patients with hand dermatitis having a history of AD (Petersen, et al., Br J Dermatol, 2014, 171:1428-1433). Similarly, a history of AD is present in approximately 14% of patients with foot dermatitis (Agner, et al., J Eur Acad Dermatol Venereol, 2015, 29:2417-2422). Other atopic conditions such as allergic rhinitis and asthma have also been associated with atopic hand and foot dermatitis (Scalone, et al., Br J Dermatol, 2015, 172:187-195).

Atopic hand and foot dermatitis (also known as atopic hand and foot eczema) presents with redness, infiltration, scaling, vesicles, areas of hyperkeratosis, and cracks (fissures) (Coenraads, N Engl J Med, 2012, 367:1829-1837). Lesions are associated with significant itching and pain. The morphology tends to evolve over time and many patients can have a mixed presentation. In the majority of patients, the same morphological subtype is found on the hands and feet (Brans, et al., Contact Dermatitis, 2015, 73:100-107). The disease tends to be chronic and recalcitrant with a substantial impact on quality of life (QoL), comparable with other skin diseases like psoriasis (Agner, et al., Contact Dermatitis, 2008, 59:43-47). Chronic hand dermatitis has also been associated with significant detrimental effect on work productivity, activity impairment, and heath care costs (Fowler, et al., J Am Acad Dermatol, 2006, 54:448-457). It has been shown that 65% of patients with severe hand dermatitis reported loss of productivity at work, with an average of 10.1 days per patient per month (Politiek, et al., Contact Dermatitis, 2016, 75:67-76). Moreover, in certain occupations like hairdressers, bakers, and machine workers, up to 18% of patients had to change jobs due to hand dermatitis (Meding, et al., Contact Dermatitis, 2005, 65:3-12).

It is known that severe atopic hand and foot dermatitis can be particularly difficult to treat. The management of hand and foot dermatitis is based upon prevention and avoidance strategies, reducing exposure to irritants and regular use of emollients. Step therapy is used with an approach to initiate treatment with topicals and then progress to systemics in case of inadequate response to topicals (Diepgen, et al., Contact Dermatitis, 2007, 57:203-210). Short courses of topical corticosteroids (TCS) are recommended as first line treatment to control flares. Long term application of TCS comes with risk of skin atrophy, dyspigmentation, acneiform eruptions, and risks associated with systemic absorption (e.g., growth retardation, hypothalamic-pituitary axis effects, etc.). Treatment guidelines for hand dermatitis of any etiology recommend continuous long-term treatment beyond 6 weeks be performed only when necessary and under careful medical supervision. Topical calcineurin inhibitors may be considered for patients with AD of hand and foot who require long-term need for treatment, although evidence for their efficacy is limited. The low penetration/permeation of topical agents through the skin of the hands and feet explains the limited efficacy of topical anti-inflammatory agents. Overnight occlusion may be advocated to allow for sufficient penetration of topical anti-inflammatory drugs. However, in the long-term, such measures are frequently unpractical and cumbersome for patients. There are currently no systemic treatments approved in the United States specifically indicated for hand and foot atopic dermatitis.

### SUMMARY

In one aspect, methods for treating atopic dermatitis of the hand and/or foot are provided. In some embodiments, the method comprises:
selecting a subject having moderate-to-severe atopic dermatitis of the hand and/or foot, wherein the subject has a baseline hand and foot Investigator's Global Assessment (IGA) overall score ≥3, and wherein the patient has a history of inadequate response of atopic hand and/or foot dermatitis to topical medication or is a patient for whom topical treatments of atopic hand and/or foot dermatitis is medically inadvisable; and
administering to the subject one or more doses of an interleukin-4 receptor (IL-4R) antagonist.

In some embodiments, the method comprises:
selecting a subject having moderate-to-severe AD of the hand and/or foot, wherein the subject is selected on the basis of having an mTLSS for hand and foot ≥16; and
administering to the subject one or more doses of an interleukin-4 receptor (IL-4R) antagonist.

In some embodiments, the IL-4R antagonist is an anti-IL-4R antibody, or an antigen-binding fragment thereof, e.g., comprising one or more CDRs, HCVR, and/or LCVR sequences set forth in Table 9. In some embodiments, the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

In some embodiments, the subject has a baseline IGA hand and foot overall score of 4. In some embodiments, the subject is inadequately responsive to treatment with a topical corticosteroid (TCS) of medium or higher potency. In some embodiments, the subject has a history of prior use of one or more systemic immunosuppressants.

In some embodiments, the subject has a baseline hand and foot peak Pruritus Numerical Rating Score (NRS) ≥4.

In some embodiments, the subject is an adult. In some embodiments, the subject is an adult who has had chronic atopic dermatitis of the hand and/or foot for at least 3 years.

In some embodiments, the subject is an adolescent. In some embodiments, the subject is an adolescent who has had chronic atopic dermatitis of the hand and/or foot for at least one year.

In some embodiments, the subject does not have irritant contact dermatitis or allergic contact dermatitis.

In some embodiments, the subject does not have atopic dermatitis lesions on parts of the body other than the hands and/or feet. In some embodiments, the subject has mild atopic dermatitis of the body other than the hands and/or feet. In some embodiments, the subject has moderate-to-severe atopic dermatitis of the body other than the hands and/or feet.

In some embodiments, the subject has a Modified Total Lesion Sign Score (mTLSS) for hand and foot score ≥16, e.g., ≥20 or ≥24. In some embodiments, the subject has a baseline hand and foot area of involvement of atopic dermatitis of at least 24%.

In some embodiments, the subject has a Body Surface Area Involvement of Atopic Dermatitis (BSA) score <10% and/or an Eczema Area and Severity Index (EASI) score <21.

In some embodiments, the subject has chronic dry fissured hand and/or foot AD. In some embodiments, the subject has hyperkeratotic hand and/or foot AD. In some embodiments, the subject has dyshidrotic hand and/or foot AD.

In some embodiments, the IL-4R antagonist is administered at a dose of about 50 mg to about 600 mg. In some embodiments, the IL-4R antagonist is administered as an initial dose of 100-600 mg followed by one or more subsequent doses of 50-300 mg. In some embodiments, the IL-4R antagonist is subcutaneously administered as an initial dose of 600 mg followed by one or more subsequent doses of 300 mg. In other embodiments, the IL-4R antagonist is subcutaneously administered as an initial dose of 300 mg followed by one or more subsequent doses of 300 mg. In some embodiments, the IL-4R antagonist is subcutaneously administered as an initial dose of 400 mg followed by one or more subsequent doses of 200 mg. In other embodiments, the IL-4R antagonist is subcutaneously administered as an initial dose of 200 mg followed by one or more subsequent doses of 200 mg. In some embodiments, each subsequent dose is administered one week or two weeks after the immediately preceding dose.

In some embodiments, the subject is an adolescent having a baseline weight 60 kg, and the IL-4R antagonist is subcutaneously administered as an initial dose of 600 mg followed by one or more subsequent doses of 300 mg Q2W.

In some embodiments, the subject is an adolescent having a baseline weight <60 kg, and the IL-4R antagonist is subcutaneously administered as an initial dose of 400 mg followed by one or more subsequent doses of 200 mg Q2W.

In some embodiments, the subject is an adult and wherein the IL-4R antagonist is subcutaneously administered as an initial dose of 600 mg followed by one or more subsequent doses of 300 mg Q2W.

In some embodiments, the IL-4R antagonist is administered for at least 16 weeks.

In some embodiments, the IL-4R antagonist is administered in combination with an emollient. In some embodiments, the IL-4R antagonist is administered in combination with a topical AD medication. In some embodiments, the topical AD medication is a TCS.

In some embodiments, treatment with the IL-4R antagonist results in the subject achieving an IGA hand and foot score of 0 or 1 by Week 16 after administration of a first dose of the IL-4R antagonist.

In some embodiments, treatment with the IL-4R antagonist results in an improvement selected from the group consisting of:
a ≥4-point reduction in hand and foot peak Pruritis NRS, relative to a baseline hand and foot peak Pruritus NRS score for the subject, by Week 16 after administration of a first dose of the IL-4R antagonist;
a reduction in mTLSS for hand and foot score of at least 50%, relative to a baseline mTLSS for hand and foot score for the subject, by Week 16 after administration of a first dose of the IL-4R antagonist;
a reduction in hand and foot peak Pruritus NRS score of at least 50%, relative to a baseline hand and foot peak Pruritus NRS score for the subject, by Week 16 after administration of a first dose of the IL-4R antagonist;
a ≥4-point reduction in hand and foot peak Pain NRS, relative to a baseline hand and foot peak Pain NRS score for the subject, by Week 16 after administration of a first dose of the IL-4R antagonist
a reduction in Hand Eczema Severity Index (HECSI) score of at least 50%, relative to a baseline HECSI score for the subject, by Week 16 after administration of a first dose of the IL-4R antagonist;
achievement of HECSI-75 by Week 16 after administration of a first dose of the IL-4R antagonist;
a reduction of at least 15% in the percent surface area of hand and foot involvement with AD, relative to a baseline percent surface area of hand and foot involvement with AD for the subject, by Week 16 after administration of a first dose of the IL-4R antagonist; and
a reduction of at least 40% in Quality of Life in Hand Eczema Questionnaire (QoLHEQ) score, relative to a baseline QoLHEQ score for the subject, by Week 16 after administration of a first dose of the IL-4R antagonist.

In some embodiments, treatment with the IL-4R antagonist decreases the need for rescue treatment.

In some embodiments, the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab.

In some embodiments, the IL-4R antagonist is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in a pen delivery device. In some embodiments, the IL-4R antagonist is contained in an autoinjector.

In another aspect, pharmaceutical compositions for treating atopic dermatitis of the hand and/or foot are provided. In some embodiments, the pharmaceutical composition comprises an interleukin-4 receptor (IL-4R) antagonist. In some embodiments, the IL-4R antagonist is an anti-IL-4R antibody, or an antigen-binding fragment thereof, e.g., comprising one or more CDRs, HCVR, and/or LCVR sequences set forth in Table 9. In some embodiments, the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

In another aspect, provided herein are interleukin-4 receptor (IL-4R) antagonists for the preparation of a medicament for the treatment of atopic dermatitis of the hand and/or foot. In some embodiments, the IL-4R antagonist is an anti-IL-4R antibody, or an antigen-binding fragment thereof, e.g., comprising one or more CDRs, HCVR, and/or LCVR sequences set forth in Table 9. In some embodiments, the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO: 6, the LCDR2 comprises the amino acid sequence LGS, and the LCDR3 comprises the amino acid sequence of SEQ ID NO:8.

Other embodiments will be apparent from a review of the ensuing detailed description.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

“Atopic dermatitis” or “AD”, as used herein, means an inflammatory skin disease characterized by intense pruritus (e.g., severe itch) and by scaly and dry eczematous lesions. The terms “hand and foot atopic dermatitis”, “hand and/or foot atopic dermatitis”, “hand and foot AD”, “hand and/or foot AD”, and “atopic dermatitis of the hand and/or foot” refer to atopic dermatitis that is localized to one or both hands and/or one or both feet. In some embodiments, a subject having atopic dermatitis localized to the hands and/or feet has one or more symptoms of atopic dermatitis elsewhere on the body. In some embodiments, a subject having atopic dermatitis localized to the hands and/or feet does not have symptoms of atopic dermatitis (e.g., lesions) elsewhere on the body.

As used herein, the term “subject in need thereof” refers to a human or a non-human animal having atopic dermatitis of the hand and/or foot (e.g., moderate-to-severe hand and/or foot AD, or severe hand and/or foot AD). In some embodiments, the term “a subject in need thereof” refers to an adult patient. In some embodiments, the term “a subject in need thereof” refers to an adolescent patient who is ≥12 and <18 years of age. The terms “subject” and “patient” are used interchangeably herein.

The term “TCS,” as used herein, includes group I, group II, group III and group IV topical corticosteroids. According to the Anatomical Therapeutic Classification System of World Health Organization, the corticosteroids are classified as weak (group I), moderately potent (Group II) and potent (Group III) and very potent (Group IV), based on their activity as compared to hydrocortisone. Group IV TCS (very potent) are up to 600 times as potent as hydrocortisone and include clobetasol propionate and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent; also referred to interchangeably herein as “medium potency”) are 2 to 25 times as potent as hydrocortisone and include, but are not limited to, clobetasone butyrate, and triamcinolone acetonide. Group I TCS (mild; also referred to interchangeably herein as “low potency”) includes hydrocortisone.

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, methods for treating atopic dermatitis (AD) of the hand and/or foot or improving a hand and/or foot AD-associated parameter in a subject are provided. In some embodiments, the methods comprise administering to a subject having moderate-to-severe hand and/or foot AD one or more doses of an interleukin-4 receptor (IL-4R) antagonist.

In some embodiments, a method for treating a subject having hand and/or foot AD comprises:
(a) selecting a subject on the basis of having one or more of the following characteristics:
   (i) a baseline IGA hand and foot score ≥3 (e.g., an IGA hand and foot score of 4);
   (ii) a baseline mTLSS hand and foot score ≥16;
   (iii) a baseline hand and foot peak Pruritus NRS (e.g., a hand and foot peak Pruritus NRS score ≥6);
   (iv) a baseline hand and foot peak Pain NRS ≥6;
   (v) a baseline sleep NRS ≥5; and/or
   (vi) a baseline hand and foot Area Involvement of Atopic Dermatitis of at least 24%, e.g., at least 25%, at least 30%, at least 35%, or more);
(b) administering to the subject identified in step (a) one or more doses of an IL-4R antagonist.

In some embodiments, a method for treating a subject having hand and/or foot AD comprises:
(a) selecting a subject on the basis of having one or more of the following characteristics:
   (i) a baseline IGA hand and foot score ≥3 (e.g., an IGA hand and foot score of 4);
   (ii) a baseline mTLSS hand and foot score ≥16;
   (iii) a baseline hand and foot peak Pruritus NRS (e.g., a hand and foot peak Pruritus NRS score ≥6);
   (iv) a baseline hand and foot peak Pain NRS ≥6;
   (v) a baseline sleep NRS ≥5; and/or
   (vi) a baseline hand and foot Area Involvement of Atopic Dermatitis of at least 24%, e.g., at least 25%, at least 30%, at least 35%, or more);
(b) selecting a subject identified in step (a) on the basis of having an overall BSA score <25% (e.g., <20%, <15%, or <10%), having an overall IGA ≤2, and/or having an overall EASI <21 (e.g., <16); and
(c) administering to the subject identified in step (b) one or more doses of an IL-4R antagonist.

In some embodiments, the subject has chronic hand and/or foot dermatitis. In some embodiments, the subject has chronic hand and/or foot dermatitis diagnosed at least 3 years prior to the start of treatment for patients years old, or at least 1 year prior to the start of treatment for patients ≥12 to <18 years old.

In some embodiments, the subject has moderate to severe atopic dermatitis of the hand(s) only. In some embodiments, the subject has moderate to severe atopic dermatitis of the foot/feet only. In some embodiments, the subject has moderate to severe atopic dermatitis of both hand(s) and foot/feet.

In some embodiments, the subject has at least two hand/foot anatomical areas with moderate to severe disease, e.g., both hands, one hand and one foot, or both feet. In some embodiments, the extent of disease in the hand/foot anatomical areas is assessed by IGA hand and foot score, e.g., as assessed by the IGA for hand and foot instrument in Table 2. In some embodiments, a subject to be treated has a baseline IGA hand and foot score ≥3, e.g., an IGA hand and foot score of 3 or an IGA hand and foot score of 4. In some embodiments, a subject to be treated has a baseline IGA hand and foot score ≥3 (i.e., an IGA hand and foot score of 3 or 4) separately for at least 2 hand/foot anatomical areas. In some embodiments, the extent of disease in the hand/foot anatomical areas is assessed by Modified Total Lesion Sign Score (mTLSS) for hands and feet. In some embodiments, a subject to be treated has a baseline mTLSS hand and foot score ≥16. In some embodiments, a subject to be treated has a baseline score of 2 or 3 for each of the mTLSS features erythema, scaling/flaking, lichenification, vesiculation/erosion, edema, and fissures in the total mTLSS hand score. In some embodiments, a subject to be treated has a baseline score of 2 or 3 for each of the mTLSS features erythema, scaling/flaking, lichenification, vesiculation/erosion, edema, and fissures in the total mTLSS foot score. In some embodiments, a subject to be treated has a baseline score of 2 or 3 for each of the mTLSS features erythema, scaling/flaking, lichenification, vesiculation/erosion, edema, and fissures in the total mTLSS hand and foot score.

In some embodiments, the subject has a baseline hand and foot peak pruritus Numeric Rating Scale (NRS) score ≥4. In some embodiments, the subject has a baseline hand and foot peak pruritus NRS score ≥6.

In some embodiments, a subject to be treated according to the methods disclosed herein is a subject who has moderate-to-severe hand and/or foot AD that is inadequately responsive to topical therapies (e.g., TCS with or without topical calcineurin inhibitors (TCIs)) or for whom topical therapy is inadvisable (e.g., due to intolerance, adverse side effects, or safety risks). In some embodiments, the subject is inadequately responsive to high potency TCS or is a subject for whom high potency TCS is inadvisable. As used herein, “inadequate response” to topical therapy refers to a failure to achieve and maintain remission or a low disease activity state as per clinical judgment of treating physician despite treatment with a daily regimen of TCS of medium to higher potency (±topical calcineurin inhibitor [TCI] as appropriate), applied for at least 28 days or for the maximum duration recommended by the product prescribing information (e.g., 14 days for super-potent TCS), whichever is shorter.

In some embodiments, a subject is considered “inadequately responsive” to topical therapy if the subject has a recent history (e.g., within 3 months or within 6 months) of treatment with a systemic immunosuppressant (e.g., cyclosporine, methotrexate, systemically administered corticosteroids, alitretinoin, etc.) for atopic hand and/or foot dermatitis.

In some embodiments, the subject is one for whom topical therapy (e.g., high potency TCS) is inadvisable. In some embodiments, the subject exhibits an intolerance to topical therapy, e.g., TCS such as high potency TCS. In some embodiments, the subject has a history of exhibiting adverse side effects or safety risks from the topical therapy, e.g., TCS such as high potency TCS. Adverse side effects or safety risks from treatment of atopic hand and/or foot dermatitis are those that outweigh the potential treatment benefits and include intolerance to treatment, hypersensitivity reactions, significant skin atrophy of hand and feet, and systemic effects, as assessed by the investigator or by the patient's treating physician.

In some embodiments, the subject does not have allergic contact dermatitis. In some embodiments, the subject does not have irritant contact dermatitis. In some embodiments, the subject does not have protein contact dermatitis. In some embodiments, the subject undergoes patch testing (e.g., with a standard series of allergens, e.g., an American Contact Dermatitis Society core allergen series, a European Baseline Series, an Australian baseline series, a patch test panel, or a thin-layer rapid use epicutaneous (TRUE) patch test) prior to the start of treatment to confirm that the subject does not have allergic contact dermatitis. In some embodiments, a subject to be treated is selected on the basis of not having allergic contact dermatitis, or is selected on the basis of having a negative patch test to confirm that the subject does not have allergic contact dermatitis.

In some embodiments, the subject does not have lesions of atopic dermatitis on parts of the body other than hands and/or feet. In some embodiments, the subject experiences flares of AD lesions on parts of the body other than hands and/or feet. In some embodiments, the subject has AD lesions on parts of the body other than hands and/or feet (e.g., over 20%, 30%, 40%, 50%, 60%, 70%, or 80% of the body other than hands and/or feet). In some embodiments, a subject having atopic hand and/or foot dermatitis also has AD lesions on parts of the body other than hands and/or feet, but has a low overall BSA score across the entire body (e.g., less than 20%, less than 15%, or less than 10%). In some embodiments, a subject having atopic hand and/or foot dermatitis also has AD lesions on parts of the body other than hands and/or feet, but has a low whole-body EASI (i.e., overall EASI across the entire body), e.g., an EASI <21, or EASI <16. In some embodiments, a subject having atopic hand and/or foot dermatitis also has AD lesions on parts of the body other than hands and/or feet, but has a low whole-body IGA (i.e., overall IGA across the entire body), e.g., an IGA ≤2 or IGA <2.

In some embodiments, a subject to be treated has a BSA score <10%. In some embodiments, a subject to be treated has an EASI <21. In some embodiments, a subject to be treated has an IGA ≤2. In some embodiments, a subject to be treated has a BSA score <10% and an EASI <21. In some embodiments, a subject to be treated has a BSA score <10% and an IGA ≤2. In some embodiments, a subject to be treated has a BSA score <10%, an EASI <21, and an IGA ≤2.

In some embodiments, the subject has chronic dry fissured hand and/or foot AD. In some embodiments, the subject has hyperkeratotic hand and/or foot AD. In some embodiments, the subject has dyshidrotic hand and/or foot AD.

In some embodiments, the subject is ≥12 years old. In some embodiments, the subject is an adult. In some embodiments, the subject is an adolescent ≥12 and <17 years of age.

In some embodiments, a subject to be treated has, or has had, a concomitant type 2 inflammatory condition. As used herein, a “type 2 inflammation condition” is a disease, disorder, or condition associated with a T helper 2 (Th2)-mediated immune response (Gandhi, et al., Nat Rev Drug Discov., 2016, 15(1):35-50). Non-limiting examples of type 2 inflammatory conditions include asthma, chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), atopic conjunctivitis, atopic dermatitis, vasculitis, cystic fibrosis (CF), chronic obstructive pulmonary disease (COPD), chronic rhinosinusitis with nasal polyps (CRSwNP), aspirin hypersensitivity, non-steroidal anti-inflammatory drug (NSAID) hypersensitivity (e.g., NSAIDs Exacerbated Respiratory Disease, or NSAID-ERD), perennial allergic rhinitis (PAR), chronic eosinophilic pneumonia (CEP) and exercise induced bronchospasm. In some embodiments, the subject has a concomitant atopic disease or condition selected from the group consisting of food allergy, atopic dermatitis, asthma, chronic rhinosinusitis, allergic rhinitis, or allergic conjunctivitis.

In some embodiments, treatment with an IL-4R antagonist improves, alleviates, or reduces one or more symptoms of hand and/or foot AD in a subject, including but not limited to pruritus, xerosis (skin dryness), eczematous lesions, erythema, papulation, edema, oozing/crusting, excoriation, lichenification, sleep disturbance, anxiety, and depression.

In some embodiments, treatment with an IL-4R antagonist improves one or more AD-associated parameters in a subject. Examples of “AD-associated parameters” include, but are not limited to: Investigators Global Assessment (IGA) of hand and foot; Modified Total Lesion Sign Score (mTLSS) for hands and feet; hand and foot Pruritus Numerical Rating Scale (NRS); hand and foot skin pain NRS; sleep NRS; hand and foot Area Involvement of Atopic Dermatitis; Dermatology Life Quality Index (DLQI); Patient Global Impression of Severity (PGIS); Patient Global Impression of Change (PGIC); Hospital Anxiety and Depression Scale (HADS); Hand Eczema Severity Index (HECSI); Quality of Life in Hand Eczema Questionnaire (QoLHEQ); Patient Oriented Eczema Measure (POEM); Eczema Area and Severity Index (EASI); Investigators Global Assessment (IGA); Body Surface Area Involvement of Atopic Dermatitis (BSA); Patient-Assessed EQ-5D; and Work Productivity and Activity Impairment Questionnaire Plus Classroom Impairment Questionnaire (WPAI+CIQ). An “improvement in an AD-associated parameter” means an improvement (e.g., decrease) from baseline of one or more of the parameters (e.g., IGA of hand and foot, mTLSS for hands and feet, hand and foot pruritus NRS, hand and foot skin pain NRS, etc.) The term “baseline,” as used with respect to an AD-associated parameter, means the numerical value of the AD-associated parameter for a subject prior to or at the onset of administration of a pharmaceutical composition as disclosed herein. In some embodiments, the AD-associated parameter is a measurement (e.g., questionnaire) that is specific for assessing the hands and/or feet. In some embodiments, the AD-associated parameter is a measurement (e.g., questionnaire) that is based on general AD (i.e., is not specific for only the hands and/or feet).

To determine whether an AD-associated parameter has “improved,” the parameter is quantified at baseline and at one or more time points after administration of the pharmaceutical composition of the present disclosure. For example, an AD-associated parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular time point following initiation of treatment and the value of the parameter at baseline is used to establish whether there has been an “improvement” (e.g., a decrease) in the AD associated parameter. AD-associated parameters are described in US Patent Publication No. US 2014/0072583, incorporated herein in its entirety.

In some embodiments, an AD-associated parameter is assessed by the patient. In some embodiments, an AD-associated parameter is assessed by a physician or by a caregiver.

In some embodiments, a subject having hand and/or foot AD is selected for treatment on the basis of having one or more of the following characteristics:
(i) a baseline IGA hand and foot score ≥3 (e.g., an IGA hand and foot score of 4);
(ii) a baseline mTLSS hand and foot score ≥16;
(iii) a baseline hand and foot peak Pruritus NRS (e.g., a hand and foot peak Pruritus NRS score ≥6);
(iv) a baseline hand and foot peak Pain NRS ≥6;
(v) a baseline sleep NRS ≥5; and/or
(vi) a baseline hand and foot Area Involvement of Atopic Dermatitis of at least 24%, e.g., at least 25%, at least 30%, at least 35%, or more).

In some embodiments, a subject having hand and/or foot AD is selected for treatment on the basis of having one or more of the above characteristics (i) through (vi) and is further selected on the basis of having an overall (i.e., entire body) BSA score <25% (e.g., <20%, <15%, or <10%) and/or having an overall EASI <21 (e.g., <16).

In some embodiments, a subject having hand and/or foot AD is selected for treatment on the basis of having a baseline mTLSS hand and foot score ≥16. In some embodiments, a subject having hand and/or foot AD is selected for treatment on the basis of having a baseline mTLSS hand and foot score ≥16, and a baseline hand and foot Area Involvement of Atopic Dermatitis of at least 24%. In some embodiments, a subject having hand and/or foot AD is selected for treatment on the basis of having a baseline mTLSS hand and foot score ≥16, and one or more of a baseline hand and foot peak Pruritus NRS ≥4, a baseline hand and foot peak Pain NRS ≥6, or a baseline sleep NRS ≥5.

In some embodiments, a subject having hand and/or foot AD is selected for treatment on the basis of having a baseline mTLSS hand and foot score ≥16, and an overall BSA score <25%. In some embodiments, a subject having hand and/or foot AD is selected for treatment on the basis of having a baseline mTLSS hand and foot score ≥16, a baseline hand and foot Area Involvement of Atopic Dermatitis of at least 24%, and an overall BSA score <25%. In some embodiments, a subject having hand and/or foot AD is selected for treatment on the basis of having a baseline mTLSS hand and foot score ≥16; one or more of a baseline hand and foot peak Pruritus NRS ≥4, a baseline hand and foot peak Pain NRS ≥6, or a baseline sleep NRS ≥5; and an overall BSA score <25%.

In some embodiments, treatment with an IL-4R antagonist according to the methods of the present disclosure results in an improvement in IGA hand and foot score for the subject relative to baseline. Methods for determining an IGA hand and foot score for a subject are described in the Examples section below and in Table 2. In some embodiments, a subject to be treated has a baseline IGA hand and foot score ≥3 (e.g., an IGA hand and foot score of 3 or an IGA hand and foot score of 4). In some embodiments, a subject is selected for treatment on the basis of having a baseline IGA hand and foot score ≥3 (e.g., an IGA hand and foot score of 3 or an IGA hand and foot score of 4). In some embodiments, treatment with an IL-4R antagonist results in a reduction from baseline in IGA hand and foot score ≥3 (e.g., from a baseline IGA hand and foot score ≥3 or a baseline IGA hand and foot score=4) of at least 1 point by week 3, week 4, week 8, week 12, or week 16 after administration of the first dose of the IL-4R antagonist. In some embodiments, treatment with an IL-4R antagonist results in a reduction from baseline (e.g., from an IGA hand and foot score ≥3 or an IGA hand and foot score=4) to an IGA hand and foot score of 0 or 1 by week 3, week 4, week 8, week 12, or week 16 after administration of the first dose of the IL-4R antagonist.

In some embodiments, treatment with an IL-4R antagonist according to the methods of the present disclosure results in an improvement in mTLSS hand and foot score for the subject relative to baseline. Methods for determining an mTLSS hand and foot score for a subject are described in the Examples section below and in Table 1. In some embodiments, a subject to be treated has a baseline mTLSS hand and foot score ≥16. In some embodiments, a subject is selected for treatment on the basis of having a baseline mTLSS hand and foot score ≥16. In some embodiments, treatment with an IL-4R antagonist results in a reduction from baseline in mTLSS hand and foot score of at least 2 points, at least 3 points, at least 4 points, at least 5 points or more by week 3, week 4, week 8, week 12, or week 16 after administration of the first dose of the IL-4R antagonist. In some embodiments, treatment with an IL-4R antagonist results in a reduction of at least 30%, at least 40%, at least 50%, at least 60%, at least 70%, at least 75%, at least 80%, or at least 90% from baseline in an mTLSS hand and foot score by week 3, week 4, week 8, week 12, or week 16 after administration of the first dose of the IL-4R antagonist.

In some embodiments, treatment with an IL-4R antagonist according to the methods of the present disclosure results in an improvement in hand and foot peak Pruritus NRS score for the subject relative to baseline. Methods for determining a hand and foot peak Pruritus NRS score for a subject are described in the Examples section below. In some embodiments, the hand and foot peak Pruritus NRS score is a weekly average of daily hand and foot peak Pruritus NRS scores. In some embodiments, a subject to be treated has a baseline hand and foot peak Pruritus NRS In some embodiments, a subject is selected for treatment on the basis of having a baseline hand and foot peak Pruritus NRS In some embodiments, treatment with an IL-4R antagonist results in a reduction from baseline in hand and foot peak Pruritus NRS score of at least 1 point, 2 points, 3 points, or 4 points by week 3, week 4, week 8, week 12, or week 16 after administration of the first dose of the IL-4R antagonist. In some embodiments, treatment with an IL-4R antagonist results in a reduction of at least 30%, at least 40%, at least 50%, at least 60%, at least 70%, at least 75%, at least 80%, or at least 90% from baseline in hand and foot peak Pruritus NRS score by week 3, week 4, week 8, week 12, or week 16 after administration of the first dose of the IL-4R antagonist.

In some embodiments, treatment with an IL-4R antagonist according to the methods of the present disclosure results in an improvement in a hand and foot peak Pain NRS score for the subject relative to baseline. Methods for determining a hand and foot peak Pain NRS score for a subject are described in the Examples section below. In some embodiments, the hand and foot peak Pain NRS score is a weekly average of daily hand and foot peak Pain NRS scores. In some embodiments, a subject to be treated has a baseline hand and foot peak Pain NRS ≥6. In some embodiments, a subject is selected for treatment on the basis of having a baseline hand and foot peak Pain NRS ≥6. In some embodiments, treatment with an IL-4R antagonist results in a reduction from baseline in hand and foot peak Pain NRS score of at least 1 point, 2 points, 3 points, or 4 points by week 3, week 4, week 8, week 12, or week 16 after administration of the first dose of the IL-4R antagonist. In some embodiments, treatment with an IL-4R antagonist results in a reduction of at least 30%, at least 40%, at least 50%, at least 60%, at least 70%, at least 75%, at least 80%, or at least 90% from baseline in hand and foot peak Pain NRS score by week 3, week 4, week 8, week 12, or week 16 after administration of the first dose of the IL-4R antagonist.

In some embodiments, treatment with an IL-4R antagonist according to the methods of the present disclosure results in an improvement in a sleep NRS score for the subject relative to baseline. Methods for determining a sleep NRS score for a subject are described in the Examples section below. In some embodiments, the sleep NRS score is a weekly average of daily sleep NRS scores. In some embodiments, a subject to be treated has a baseline sleep NRS ≥5. In some embodiments, a subject is selected for treatment on the basis of having a baseline sleep NRS ≥5. In some embodiments, treatment with an IL-4R antagonist results in a reduction from baseline in sleep NRS score of at least 1 point by week 3, week 4, week 8, week 12, or week 16 after administration of the first dose of the IL-4R antagonist. In some embodiments, treatment with an IL-4R antagonist results in a reduction of at least 30%, at least 40%, at least 50%, at least 60%, at least 70%, at least 75%, at least 80%, or at least 90% from baseline in sleep NRS score by week 3, week 4, week 8, week 12, or week 16 after administration of the first dose of the IL-4R antagonist.

In some embodiments, treatment with an IL-4R antagonist according to the methods of the present disclosure results in an improvement in a hand and foot Area Involvement of Atopic Dermatitis score for the subject relative to baseline (e.g., a decrease in the percent of surface area of hand and foot involvement with AD). In some embodiments, a subject to be treated has a baseline hand and foot Area Involvement of Atopic Dermatitis of at least 24%, e.g., at least 25%, at least 30%, at least 35%, or more. In some embodiments, a subject is selected for treatment on the basis of having a baseline hand and foot Area Involvement of Atopic Dermatitis of at least 24%, e.g., at least 25%, at least 30%, at least 35%, or more. In some embodiments, treatment with an IL-4R antagonist results in a reduction of at least 10%, at least 15%, at least 20%, at least 25%, at least 30%, at least 40%, at least 50%, or at least 60% from baseline in the surface area of hand and foot affected with AD by week 3, week 4, week 8, week 12, or week 16 after administration of the first dose of the IL-4R antagonist.

In some embodiments, treatment with an IL-4R antagonist according to the methods of the present disclosure results in an improvement in a Hand Eczema Severity Index (HECSI) score for the subject relative to baseline. Methods for determining a HECSI score for a subject are described in the Examples section below. In some embodiments, a subject to be treated has a baseline HECSI score ≥46, e.g., least 50, at least 55, at least 60, at least 65, at least 70, or higher. In some embodiments, a subject is selected for treatment on the basis of having a baseline HECSI score ≥46, e.g., least 50, at least 55, at least 60, at least 65, at least 70, or higher. In some embodiments, treatment with an IL-4R antagonist results in a reduction from baseline in HECSI score of at least 15, 20, 25, 30 or more points by week 3, week 4, week 8, week 12, or week 16 after administration of the first dose of the IL-4R antagonist. In some embodiments, treatment with an IL-4R antagonist results in a reduction of at least 30%, at least 40%, at least 50%, at least 60%, at least 70%, at least 75%, at least 80%, or at least 90% from baseline in HECSI score by week 3, week 4, week 8, week 12, or week 16 after administration of the first dose of the IL-4R antagonist. In some embodiments, treatment with an IL-4R antagonist results in the subject achieving HECSI-50 (i.e., at least 50% reduction in HECSI score) by week 3, week 4, week 8, week 12, or week 16 after administration of the first dose of the IL-4R antagonist. In some embodiments, treatment with an IL-4R antagonist results in the subject achieving HECSI-75 (i.e., at least 75% reduction in HECSI score) by week 3, week 4, week 8, week 12, or week 16 after administration of the first dose of the IL-4R antagonist. In some embodiments, treatment with an IL-4R antagonist results in the subject achieving HECSI-90 (i.e., at least 90% reduction in HECSI score) by week 3, week 4, week 8, week 12, or week 16 after administration of the first dose of the IL-4R antagonist.

In some embodiments, treatment with an IL-4R antagonist according to the methods of the present disclosure results in an improvement in health-related quality of life for the subject relative to baseline, e.g., as measured by DLQI or QoLHEQ. In some embodiments, treatment with an IL-4R antagonist results in an improvement of at least 30%, at least 40%, at least 50%, at least 60%, at least 70%, at least 75%, at least 80%, or at least 90% from baseline in a health-related quality of life measure by week 3, week 4, week 8, week 12, or week 16 after administration of the first dose of the IL-4R antagonist.

In some embodiments, treatment with an IL-4R antagonist reduces the need for a rescue treatment (e.g., for AD flares, for lesions persisting or worsening under daily treatment, or for intolerable symptoms). In some embodiments, treatment with the IL-4R antagonist decreases the need for a topical rescue treatment (e.g., topical corticosteroids such as medium-potency TCS or high potency TCS, TCI, crisaborole, or topical JAK inhibitor). In some embodiments, treatment with the IL-4R antagonist decreases the need for a systemic rescue treatment (e.g., systemic corticosteroids or systemic immunosuppressants).

### Interleukin-4 Receptor Antagonists

In some embodiments, the methods of the present disclosure comprise administering to a subject in need thereof (e.g., a subject having moderate-to-severe atopic dermatitis of the hand and/or foot) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V H) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V L domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (VI) V_{H}-C_{H}2-C_{H}3; V_{H}-C_{L}; V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein, et al., 2012, mAbs, 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor, et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (K D) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, NJ). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises the amino acid sequence GFTFRDYA (SEQ ID NO:3), the HCDR2 comprises the amino acid sequence ISGSGGNT (SEQ ID NO:4), the HCDR3 comprises the amino acid sequence AKDRLSITIRPRYYGLDV (SEQ ID NO:5), the LCDR1 comprises the amino acid sequence QSLLYSIGYNY (SEQ ID NO:6), the LCDR2 comprises the amino acid sequence LGS, and the LCDR3 comprises the amino acid sequence MQALQTPYT (SEQ ID NO:8).

In some embodiments, the anti-IL-4R antibody or antigen-binding fragment thereof comprises an HCDR1 comprising the amino acid sequence GFTFRDYA (SEQ ID NO:3), an HCDR2 comprising the amino acid sequence ISGSGGNT (SEQ ID NO:4), an HCDR3 comprising the amino acid sequence AKDRLSITIRPRYYGLDV (SEQ ID NO:5), an LCDR1 comprising the amino acid sequence QSLLYSIGYNY (SEQ ID NO:6), an LCDR2 comprising the amino acid sequence LGS, and an LCDR3 comprising the amino acid sequence MQALQTPYT (SEQ ID NO:8), and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141, or International Patent Publication Nos. WO2020/096381, WO 2020/182197, WO2020/239134, WO 2021/213329, WO2022/052974, WO2022/136669, or WO2022/136675, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 9 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-V_{L}-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-V_{L}); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-V_{L}); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-V_{L}); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-V_{L}), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody comprises (i) an HCVR comprising the amino acid sequence of SEQ ID NO:208 (STSA-C27-VH), SEQ ID NO:209 (STSA-C27-6-33-VH), SEQ ID NO:210 (STSA-C27-7-33-VH), SEQ ID NO:211 (STSA-C27-24-56-VH), SEQ ID NO:212 (STSA-C27-47-56-VH), SEQ ID NO:213 (STSA-C27-33-33-VH), SEQ ID NO:214 (STSA-C27-56-56-VH), SEQ ID NO:215 (STSA-C27-78-78-VH), SEQ ID NO:216 (STSA-C27-82-58-VH), SEQ ID NO:217 (STSA-C27-54-54-VH), SEQ ID NO:218 (STSA-C27-36-36-V_{H}), SEQ ID NO:219 (STSA-C27-53-53-VH), SEQ ID NO:220 (STSA-C27-67-67-VH), SEQ ID NO:221 (STSA-C27-55-55-VH), SEQ ID NO:222 (STSA-C27-59-59-VH), SEQ ID NO:223 (STSA-C27-58-58-VH), SEQ ID NO:224 (STSA-C27-52-52-VH), or SEQ ID NO:225 (STSA-027-Y2-Y2-VH); and (ii) an LCVR comprising the amino acid sequence of SEQ ID NO:226 (STSA-C27-VL), SEQ ID NO:227 (STSA-C27-6-33-VL), SEQ ID NO:228 (STSA-C27-7-33-V_{L}), SEQ ID NO:229 (STSA-C27-24-56-VL), SEQ ID NO:230 (STSA-C27-47-56-VL), SEQ ID NO:231 (STSA-C27-33-33-VL), SEQ ID NO:232 (STSA-027-56-56-VL), SEQ ID NO:233 (STSA-C27-78-78-VL), SEQ ID NO:234 (STSA-027-82-58-VL), SEQ ID NO:235 (STSA-C27-54-54-VL), SEQ ID NO:236 (STSA-C27-36-36-VL), SEQ ID NO:237 (STSA-C27-53-53-V_{L}), SEQ ID NO:238 (STSA-C27-67-67-VL), SEQ ID NO:239 (STSA-027-55-55-VL), SEQ ID NO:240 (STSA-C27-59-59-VL), SEQ ID NO:241 (STSA-027-58-58-VL), SEQ ID NO:242 (STSA-C27-52-52-VL), or SEQ ID NO:243 (STSA-C27-Y2-Y2-VL).

In some embodiments, an anti-IL-4Rα antibody comprises (i) an HCVR comprising the amino acid sequence of SEQ ID NO:244 (Y0188-1 VH), SEQ ID NO:245 (Y0188-2 VH), SEQ ID NO:246 (Y0188-3 VH), SEQ ID NO:247 (Y0188-4 VH), SEQ ID NO:248 (Y0188-6 VH), SEQ ID NO:249 (Y0188-8 VH), SEQ ID NO:250 (Y0188-9 VH), SEQ ID NO:251 (Y0188-10 VH), SEQ ID NO:252 (Y0188-14 VH), SEQ ID NO:253 (HV3-15-14 VH), SEQ ID NO:254 (HV3-48-14 VH), SEQ ID NO:255 (HV3-73*2-14 VH), SEQ ID NO:256 (HV3-72-14 VH), SEQ ID NO:257 (Y01-14 VH), SEQ ID NO:258 (162-14 VH), or SEQ ID NO:259 (VH73-14 VH); and (ii) an LCVR comprising the amino acid sequence of SEQ ID NO:260 (Y0188-1 VL), SEQ ID NO:261 (Y0188-2 VL), SEQ ID NO:262 (Y0188-3 VL), SEQ ID NO:263 (Y0188-4 VL), SEQ ID NO:264 (Y0188-6 VL), SEQ ID NO:265 (Y0188-8 VL), SEQ ID NO:266 (Y0188-9 VL), SEQ ID NO:267 (Y0188-10 VL), SEQ ID NO:268 (Y0188-14 VL), SEQ ID NO:269 (Y01-14 VL), SEQ ID NO:270 (164-14 VL), SEQ ID NO:271 (KV4-14 VL), SEQ ID NO:272 (KV1-27-14 VL), SEQ ID NO:273 (KV1-9-14 VL), SEQ ID NO:274 (KV1-NL1-14 VL), or SEQ ID NO:275 (KV1D-43-14 VL).

In some embodiments, an anti-IL-4Rα antibody used in the methods of the present disclosure can have pH-dependent binding characteristics. For example, an anti-IL-4Rα antibody for use as disclosed herein may exhibit reduced binding to IL-4Rα at acidic pH as compared to neutral pH. Alternatively, an anti-IL-4Rα antibody for use as disclosed herein may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2 and HCDR3) contained within a heavy chain variable region (HCVR) having an amino acid sequence of SEQ ID NO:1, and the three light chain CDRs (LCVR1, LCVR2, and LCVR3) contained within a light chain variable region (LCVR) having an amino acid sequence of SEQ ID NO:2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani, et al., J. Mol. Biol. 273:927-948 (1997); and Martin, et al., Proc. Natl. Acad. Sci. USA 86:9268-9272 (1989). Public databases are also available for identifying CDR sequences within an antibody.

### Pharmaceutical Compositions

In one aspect, the present disclosure provides methods that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti, et al., 1991, Pharmaceut. Res. 8:1351). Specific exemplary doses of anti-IL4R antibodies, and administration regimens involving the same, that can be used in the context of the present disclosure are disclosed elsewhere herein.

In some embodiments, an IL-4R antagonist or a pharmaceutical composition of the present disclosure is contained within a container. Thus, in another aspect, containers comprising an IL-4R antagonist or a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L.P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem. 262:4429-4432).

In some embodiments, a pharmaceutical composition comprising an anti-IL-4R antibody is administered using a drug delivery device that is a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Exemplary audible end-of-dose feedback mechanisms are described in International Publications WO2016/193346 and WO2016/193348. An exemplary needle-safety mechanism after using an auto-injector is described in International Publication WO2016/193352. An exemplary needle sheath remover mechanism for a syringe auto-injector is described in International Publication WO2016/193353. An exemplary support mechanism for supporting an axial position of a syringe is described in International Publication WO2016/193355.

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present disclosure are disclosed, e.g., in U.S. Pat. No. 8,945,559.

### Dosage and Administration

In some embodiments, an IL-4R antagonist (e.g., anti-IL-4R antibody) is administered to a subject (e.g., a subject having moderate to severe atopic hand and/or foot dermatitis) according to the methods of the present disclosure in a therapeutically effective amount. As used herein with reference to an IL-4R antagonist, the phrase “therapeutically effective amount” means an amount of IL-4R antagonist that results in one or more of: (a) an improvement in one or more AD-associated parameters (as mentioned elsewhere herein); and/or (b) a detectable improvement in one or more symptoms or indicia of atopic hand and/or foot dermatitis.

In the case of an anti-IL-4R antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In some embodiments, a therapeutically effective amount is from about 50 mg to about 600 mg, or from about 100 mg to about 600 mg, or from about 200 mg to about 600 mg. In certain embodiments, 50 mg, 75 mg, 100 mg, 125 mg, 150 mg, 200 mg, 250 mg, 300 mg, 350 mg, 400 mg, 450 mg, 500 mg, 550 mg, or 600 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial or loading dose and the one or more secondary or maintenance doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In some embodiments, one or more maintenance doses of the IL-4R antagonist are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 week, 2 weeks, 3 weeks, or 4 weeks after the immediately preceding dose. Similarly, in some embodiments involving multiple tertiary doses, each tertiary dose is administered at the same frequency as the other tertiary doses. For example, each tertiary dose may be administered to the patient 1 week, 2 weeks, 3 weeks, or 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In some embodiments, a therapeutically effective amount of an IL-4R antagonist (e.g., anti-IL-4R antibody) comprises 300 mg administered every two weeks (Q2W). In some embodiments, a therapeutically effective amount of an IL-4R antagonist (e.g., anti-IL-4R antibody) comprises a loading dose of 600 mg followed by one or more subsequent doses of 300 mg administered every two weeks (Q2W). In some embodiments, no loading dose is administered.

In some embodiments, a therapeutically effective amount of an IL-4R antagonist (e.g., anti-IL-4R antibody) comprises 200 mg administered every two weeks (Q2W). In some embodiments, a therapeutically effective amount of an IL-4R antagonist (e.g., anti-IL-4R antibody) comprises a loading dose of 400 mg followed by one or more subsequent doses of 200 mg administered every two weeks (Q2W). In some embodiments, no loading dose is administered.

In some embodiments, for a subject having moderate-to-severe atopic dermatitis of the hand and/or foot or severe AD who is to ≥12 to <18 years of age, a therapeutically effective amount of an IL-4R antagonist (e.g., anti-IL-4R antibody) comprises 200 mg administered every two weeks (Q2W), if the subject is <60 kg in weight. In some embodiments, the subject is administered a loading dose of 400 mg followed by one or more subsequent doses of 200 mg administered every two weeks (Q2W), if the subject is <60 kg in weight. In some embodiments, no loading dose is administered.

In some embodiments, for a subject having moderate-to-severe atopic dermatitis of the hand and/or foot or severe AD who is to ≥12 to <18 years of age, a therapeutically effective amount of an IL-4R antagonist (e.g., anti-IL-4R antibody) comprises 300 mg administered every two weeks (Q2W), if the subject is ≥60 kg in weight. In some embodiments, the subject is administered a loading dose of 600 mg followed by one or more subsequent doses of 300 mg administered every two weeks (Q2W), if the subject is ≥60 kg in weight. In some embodiments, no loading dose is administered.

### Combination Therapies

In some embodiments, the methods of the present disclosure comprise administering to the subject (e.g., a subject having moderate-to-severe atopic dermatitis of the hand and/or foot) an IL-4R antagonist according to the disclosure (e.g., an anti-IL-4R antibody) in combination with one or more additional therapeutic agents. In some embodiments, the additional therapeutic agent is a topical therapeutic agent, e.g., a TCS or a topical nonsteroidal medication such as a TCI, crisaborole, or topical JAK inhibitor. As used herein, the expression “in combination with” means that the topical therapy (e.g., TCS) is administered before, after, or concurrent with the IL-4R inhibitor. The term “in combination with” also includes sequential or concomitant administration of IL-4R inhibitor and the topical therapy (e.g., TCS).

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than about 10 minutes (before, after, or at the same time) of administration of the pharmaceutical composition comprising the IL-4R antagonist, or administered to the subject as a single combined dosage formulation comprising both the additional therapeutic agent and the IL-4R antagonist.

In some embodiments, the additional therapeutic agent is a TCS. In some embodiments, the TCS is a medium-potency TCS. In some embodiments, the TCS is a low-potency TCS. In some embodiments, the additional therapeutic agent is a TCI. In some embodiments, the additional therapeutic agent is crisaborole.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial Evaluating the Efficacy and Safety of Dupilumab in Adult and Adolescent Patients with Moderate-to-Severe Atopic Hand and/or Foot Dermatitis

### Study Design and Objectives

This was a global, multicenter, randomized, double-blind, parallel-group, placebo-controlled Phase 3 study investigating the efficacy and safety of dupilumab monotherapy in adult and adolescent patients with moderate-to-severe atopic hand and/or foot dermatitis (NCT04417894). The study consisted of three periods: a screening period (at least 4 weeks and up to 8 weeks), a randomized treatment period (16 weeks), and a post-treatment follow-up period (12 weeks).

Dupilumab is a fully human anti-IL-4R antibody comprising a heavy chain comprising the amino acid sequence of SEQ ID NO:9 and a light chain comprising the amino acid sequence of SEQ ID NO:10; an HCVR/LCVR amino acid sequence pair comprising SEQ ID NOs:½; and heavy and light chain CDR sequences comprising SEQ ID NOs:3-8.

This study was conducted in accordance with the provisions of the Declaration of Helsinki, the International Conference on Harmonization Good Clinical Practices guideline, and applicable regulatory requirements. The protocol was reviewed and approved by institutional review boards/ethics committees at all sites. At the screening visit, adult patients provided informed consent before any other study procedures were conducted. For adolescent patients, parents or legal guardians provided informed consent and the patients provided assent.

### Patient Population

The study population was patients with chronic, moderate-to-severe atopic hand and/or foot dermatitis inadequately responsive to medium-to-high potency TCS or in whom medium-to-high potency TCS is inadvisable. Only patients meeting the diagnostic criteria for AD were included in this study. Patients with a confirmed diagnosis of irritant contact dermatitis or allergic contact dermatitis as the predominant cause of hand and/or foot dermatitis were excluded from the study. Patients were randomized in a 1:1 ratio stratified by age (adults vs adolescents), baseline disease severity (HF-IGA score ≥3 vs 4), and geographic region (USA vs Japan vs EU) to subcutaneous dupilumab or placebo every 2 weeks (q2w) for 16 weeks.

Inclusion Criteria: A patient had to meet the following criteria to be eligible for inclusion in the study: (1) Male or female, years old at time of screening visit; (2) Patients with chronic hand and/or foot dermatitis diagnosed at least 3 years prior to the screening visit for patients years old, and at least 1 year prior to the screening visit for patients to ≥12 to <18 years old; (3) Patients with involvement of at least 2 anatomical areas at screening and baseline. These 2 anatomical areas could be both hands, 1 hand and 1 foot, or both feet. (4) Patients need to have an IGA hand and foot score of 3 or 4 (moderate-to-severe disease) at screening and baseline. Note: The investigators should assign a single IGA score based on overall impression of disease severity of hands and feet. Patients need to have overall moderate-to-severe disease in hands/feet and involvement of at least 2 anatomical areas to be eligible. (5) Patients with documented recent history (within 6 months before the screening visit) of inadequate response of atopic hand and/or foot dermatitis to topical medication(s) or for whom topical treatments of atopic hand and/or foot dermatitis is medically inadvisable (e.g., intolerance, because of important side effects or safety risks). If documentation is inadequate, potential patients may be offered a course of treatment with a daily regimen of TCS of medium or higher potency (±TCI as appropriate), applied for at least 28 days during the screening period, or for the maximum duration recommended by the product prescribing information, whichever is shorter. Patients who demonstrate inadequate response during this period, as defined above, will be eligible for inclusion in the study following appropriate washout. (6) Patients need to meet the Hanifin and Rajka criteria for diagnosis (Hanifin, 1980). (7) Baseline hand and foot peak pruritus NRS score for maximum itch intensity ≥4; (8) At least 11 (of a total of 14) applications of a topical emollient (moisturizer) during the 7 consecutive days immediately before the baseline visit (not including the day of randomization); (9) Willing and able to comply with clinic visits and study-related procedures; (10) Provide informed consent/assent signed by study patient or legally acceptable representative; (12) Patients need to have been compliant with skin protection measures through the entire duration of the screening period (minimum of 4 weeks but lasting to a maximum of 8 weeks). This includes avoidance of known irritants that are identified as causing exacerbation of one's hand and/or foot AD.

NOTES for inclusion criteria (5): Inadequate response is defined as failure to achieve and maintain remission or a low disease activity state as per clinical judgment of treating physician despite treatment with a daily regimen of TCS of medium to higher potency (±topical calcineurin inhibitor [TCI] as appropriate), applied for at least 28 days or for the maximum duration recommended by the product prescribing information (e.g., 14 days for super-potent TCS), whichever is shorter. Important side effects or safety risks from treatment of atopic hand and/or foot dermatitis are those that outweigh the potential treatment benefits and include intolerance to treatment, hypersensitivity reactions, significant skin atrophy of hand and feet, and systemic effects, as assessed by the investigator or by the patient's treating physician. Acceptable documentation includes contemporaneous chart notes that record topical medication prescription and treatment outcome, or investigator documentation based on communication with the patient's treating physician.

NOTES for inclusion criteria (6): The diagnosis of AD requires the presence of at least 3 of 4 major criteria: (a) pruritus; (b) dermatitis affecting flexural surfaces in adults; (c) chronic or relapsing dermatitis; (d) personal or family history of cutaneous or respiratory atopy. In patients who do not have AD lesions present on parts of the body other than hands and feet at time of screening, the study investigator should check for any prior history of presence of AD lesions in typical age specific distribution patterns for e.g., flexural areas (cutaneous atopy). Prior history of respiratory atopic diseases (for e.g., asthma, allergic rhinitis) should also be elicited. In addition, 3 out of 23 minor criteria need to be met. Minor criteria include: features of the so-called “atopic facies”: facial pallor or erythema, hypopigmented patches, infraorbital darkening, infraorbital folds or wrinkles, cheilitis, recurrent conjunctivitis, and anterior neck folds; triggers of atopic dermatitis: foods, emotional factors, environmental factors, and skin irritants such as wool, solvents, and sweat; complications of atopic dermatitis: susceptibility to cutaneous viral and bacterial infections, impaired cell-mediated immunity, immediate skin-test reactivity, raised serum IgE, keratoconus, anterior subcapsular cataracts; others: early age of onset, dry skin, ichthyosis, hyperlinear palms, keratosis pilaris (plugged hair follicles of proximal extremities), hand and/or foot dermatitis, nipple eczema, white dermatographism, and perifollicular accentuation.

Exclusion Criteria: The following were exclusion criteria for the study: (1) Patients with a positive patch test reaction to one or more allergens (a score of 1+ or above according to International Contact Dermatitis Research Group [ICDRG] grading scale) in either (a) the baseline patch test series, or (b) extended baseline or supplemental patch test allergens if such additional testing is conducted by the investigator, or (c) personal products if testing with such products is conducted by the investigator; which is deemed to be clinically relevant in the view of the investigator as the current cause of the hand and/or foot dermatitis. [NOTES: Patients with a documented diagnosis of allergic contact dermatitis of hands and/or feet, who have a positive patch test reaction at screening will also be excluded from the study, regardless of whether history of current skin exposure to products containing this allergen (current relevance) is present. Patients with positive reactions which are interpreted as irritant reactions based upon morphology, timing (e.g., positive at day 2 followed by a decrescendo pattern) will still be eligible for study as long as they meet other eligibility criteria for the study.] (2) Patients with a documented diagnosis of protein contact dermatitis of hands and/or feet. These are patients with occupational or non-occupational contact to proteins such as food, latex, etc., with positive prick test who present with lesions of contact urticaria or dermatitis on hands/feet. (3) Patients in whom patch testing cannot be conducted due to any reason (these include but are not limited to refusal of patient to have patch testing, inability to take patient off systemic immunosuppressants/topical AD medications for the required washout period prior to patch testing, lack of clear skin (free of lesions) on upper back and/or upper arm on which to apply patch tests, etc.). (4) Patients with documented exposure to irritants in the occupational or non-occupational (household/recreational) setting that is believed to be a predominant cause of the current hand and/or foot dermatitis as per the judgment of the investigator. (5) Treatment with dupilumab in the past. (6) Patients who have used any the following treatments within 4 weeks before the baseline visit: (a) systemic corticosteroids; (b) immunosuppressive/immunomodulating drugs (e.g., alitretinoin, cyclosporine, mycophenolate mofetil, IFN-γ, Janus kinase inhibitors, azathioprine or methotrexate); (c) phototherapy (including localized psoralen and UVA [PUVA] or narrow band ultraviolet B [UVB] on hands and/or feet). (7) Treatment with biologics, other than dupilumab, as follows: (a) any cell-depleting agents including but not limited to rituximab: within 6 months before the baseline visit, or until lymphocyte and CD 19+ lymphocyte count returns to normal, whichever is longer; (b) other biologics: within 5 half-lives (if known) or 16 weeks prior to the baseline visit, whichever is longer. (8) Treatment with TCS or TCI or crisaborole or topical JAK inhibitor within 2 weeks before the baseline visit on the hand and foot. (9) Treatment with an investigational drug within 8 weeks or within 5 half-lives (if known), whichever is longer, before the baseline visit. (10) Treatment with a live (attenuated) vaccine within 4 weeks before the baseline visit. (11) Planned or anticipated use of any prohibited medications and procedures during study treatment. (12) Known or suspected immunodeficiency, including history of invasive opportunistic infections (e.g., tuberculosis [TB], histoplasmosis, listeriosis, coccidioidomycosis, pneumocystosis, aspergillosis) despite infection resolution, or otherwise recurrent infections of abnormal frequency as judged by the investigator. (13) Known history of human immunodeficiency virus (HIV) infection or HIV seropositivity. (14) Current diagnosis of hepatitis B viral infection at the time of screening as evidenced by (a) positive hepatitis B surface antigen (HBsAg) or (b) positive total hepatitis B core antibody (HBcAb) confirmed by positive HBV DNA. (15) Current diagnosis of hepatitis C viral infection at the time of screening as evidence by (a) positive HCV Ab and (b) positive HCV RNA. (16) On current treatment for hepatic disease including but not limited to acute or chronic hepatitis, cirrhosis, or hepatic failure, or has evidence of liver disease as indicated by persistent (confirmed by repeated tests weeks apart) elevated transaminases (alanine aminotransferase [ALT] and/or aspartate aminotransferase [AST]) more than 3 times the upper limit of normal (ULN) during the screening period. (17) Presence of any 1 or more of the following abnormalities in laboratory test results at screening: (a) platelets 100×10³/μL; (b) neutrophils <1.5×10³/μL. (18) Diagnosed active endoparasitic infections. (19) Presence of skin comorbidities on hand and/or foot that may interfere with study assessments. This includes, but is not limited to palmoplantar psoriasis, palmoplantar keratoderma, lichen planus, pityriasis rubra pilaris, herpes simplex, erythema multiforme, tinea mannum/tinea pedis, scabies, granuloma annulare. (20) History of malignancy within 5 years before the baseline visit, except completely treated in situ carcinoma of the cervix, completely treated and resolved non-metastatic squamous or basal cell carcinoma of the skin. (21) Severe concomitant illness(es) that, in the investigator's judgment, would adversely affect the patient's participation in the study. Examples include, but are not limited to patients with short life expectancy, patients with uncontrolled diabetes (HbAlc≥9%), patients with cardiovascular conditions (e.g., stage III or IV cardiac failure according to the New York Heart Association classification), severe renal conditions (e.g., patients on dialysis), neurological conditions (e.g., demyelinating diseases), active major autoimmune diseases (e.g., Eosinophilic granulomatosis with polyangitis (EGPA), lupus, inflammatory bowel disease, rheumatoid arthritis, etc.), other severe endocrinological, gastrointestinal, hepato-biliary, metabolic, pulmonary or lymphatic diseases. (22) Any other medical or psychological condition (including relevant laboratory abnormalities at screening) that, in the opinion of the investigator, may suggest a new and/or insufficiently understood disease, may present an unreasonable risk to the study patient as a result of his/her participation in this clinical trial, may make patient's participation unreliable, or may interfere with study assessments. (23) History of alcohol or drug abuse within 2 years before the screening visit. (24) Patients who are committed to an institution by virtue of an order issued either by the judicial or the administrative authorities. (25) Patient is a member of the investigational team or his/her immediate family. (26) Pregnant or breastfeeding women or planning to become pregnant or breastfeed during the patient's participation in this study. (27) Women of childbearing potential (WOCBP) who are unwilling to practice highly effective contraception prior to the initial dose/start of the first treatment, during the study, and for at least 12 weeks after the last dose.

### Study Treatments

Study drug treatments were as follows:
Dupilumab, administered subcutaneously Q2W
   in adults: dupilumab 300 mg, after a loading dose of 600 mg on day 1, irrespective of body weight.
   in adolescents: body weight ≥60 kg, dupilumab 300 mg, after a loading dose of 600 mg on day 1, body weight <60 kg, dupilumab 200 mg, after a loading dose of 400 mg on day 1.
Matching placebo, administered subcutaneously Q2W

It was recommended that SC injection sites of the study drug be alternated among the different quadrants of the abdomen (avoiding navel and waist areas), upper thighs, and upper arms so that the same site was not injected for 2 consecutive injections.

Background treatment: All patients were required to apply moisturizers on their hands and feet at least twice daily during the screening period. It was recommended that patients continue the use of moisturizers throughout the study (all 28 weeks where applicable). All types of moisturizers were permitted, but patients could not initiate treatment with prescription moisturizers or moisturizers containing additives during the screening period or during the study. Patients could continue using stable doses of such moisturizers if initiated before the screening visit. It was recommended that the moisturizers used be free of additives, fragrances, perfumes, and other potentially sensitizing agents. Moreover, it was recommended that the moisturizers not contain any compound with known anti-itch effect (such as pramoxine, lidocaine, prilocaine, capsaicin etc.).

Rescue treatment: Rescue treatment for worsening of hand and/or foot AD could be provided to study patients at the discretion of the investigator in the study. The use of rescue treatment was only allowed after day 14 of the study. Investigators were required to perform an IGA for hands and feet assessment prior to starting rescue treatment and initiate rescue treatment only in patients who have an IGA hand and foot score ≥3. If possible, investigators were encouraged to consider rescue initially with topical treatment (e.g., high potency or ultra-high potency TCS, TCIs, crisaborole, or topical JAK inhibitors) and to escalate to systemic medications only for patients who did not respond adequately after at least 7 days of topical treatment. Rescue treatment for the topical therapies could be used as per prescribing information and local guidelines. Patients could continue study treatment if rescue consists of topical medications.

Investigators could also use systemic corticosteroids or non-steroidal systemic immunosuppressive drugs (alitretinoin, cyclosporine, methotrexate, mycophenolate-mofetil, azathioprine, JAK inhibitors, etc.) for rescue in patients with worsening of AD of hands and feet. Patients could continue study treatment if rescue consists of these systemic therapies, based upon discretion of investigator. All patients were asked to complete the scheduled study visits and assessments whether or not they complete study treatment and whether or not they receive rescue treatment for AD. Investigators should make every attempt to conduct efficacy and safety assessments (e.g., disease severity scores, safety laboratory tests) immediately before administering any rescue treatment.

### Outcomes Assessed

The primary endpoint for the study was the proportion of patients achieving an IGA (hand and foot) score of 0 or 1 at week 16.

The key secondary endpoint was the proportion of patients with improvement (reduction) of weekly average of daily hand and foot peak Pruritus NRS from baseline to week 16.

Other secondary endpoints for efficacy included: percent change in Modified Total Lesion Sign Score (mTLSS) for hand/foot lesions from baseline to week 16; proportion of patients with improvement (reduction) of weekly average of daily hand and foot peak Pruritus NRS from baseline to week 16; percent change from baseline to week 16 in weekly average of daily hand and foot peak Pruritus NRS; percent change from baseline to week 16 in weekly average of daily hand and foot peak Pain NRS; percent change from baseline to week 16 in weekly average of daily Sleep NRS; change from baseline to week 16 in percent surface area of hand and foot involvement with AD; percent change from baseline to week 4 in weekly average of daily hand and foot peak Pruritus NRS; proportion of patients with improvement (reduction) of weekly average of daily hand and foot peak Pruritus NRS from baseline to week 4; for patients with hand dermatitis, percent change from baseline to week 16 in Hand Eczema Severity Index (HECSI) score; for patients with hand dermatitis, proportion of patients with HECSI-75 at week 16; for patients with hand dermatitis, proportion of patients with HECSI-50 at week 16; for patients with hand dermatitis, proportion of patients with HECSI-90 at week 16; for patients with hand dermatitis, change from baseline to week 16 in Quality of Life in Hand Eczema Questionnaire (QOLHEQ); change from baseline to week 16 in Work Productivity and Impairment (WPAI) and Classroom Impairment Questionnaire (CIQ).

The secondary endpoints for safety included: incidence of treatment-emergent adverse events (TEAEs) through week 16.

The secondary endpoints for clinical pharmacology and immunogenicity included: trough concentration of functional dupilumab in serum at various time points; incidence of treatment-emergent anti-drug antibody (ADA) and titer over time.

Procedures for assessing efficacy (e.g., using IGA for hands and feet, mTLSS, Daily hand and foot peak Pruritus NRS, Daily hand and foot peak Pain NRS, or other methods of assessment) are described below, or are described in WO 2021/026205, incorporated by reference herein.

Modified Total Lesion Sign Score for Hands and Feet: The Modified Total Lesion Sign Score (mTLSS) is adapted for hands and feet; which has been previously used in registrational studies in hand dermatitis (Ruzicka, et al., Br J Dermatol, 2008, 158:808-817). Investigators assess the severity of signs of disease on hands and feet on this scale; this assessment is limited to only the hands and feet and is not influenced by severity of AD lesions on other parts of the body. The mTLSS score is assessed at screening, baseline, and on specified days during and/or after treatment.

The mTLSS is assessed by assigning a score to each of the 6 features (erythema, scaling/flaking, lichenification, vesiculation/erosion, edema, fissures) on a scale of 0 to 3 (0=absent, 1=mild, 2=moderate, 3=severe) based on the morphological descriptions of severity provided in Table 1 below. A separate score is assigned for hands and for feet. Total hand mTLSS is from 0-18. Total foot mTLSS is from 0-18. A total mTLSS score (from 0-36) is calculated as the sum of the total hand score and the total foot score.

TABLE 1
mTLSS Scoring for Hand and Foot

| Feature | Description of Severity |
|---|---|
| Erythema | 0 = absent |
| | 1 = barely perceptible (pink) redness (mild) |
| | 2 = clearly perceptible (dull red) OR prominent (deep/dark red) redness in limited areas |
| | (moderate) |
| | 3 = prominent (deep/dark red) redness over widespread areas (severe) |
| Scaling/Flaking^{a} | 0 = absent |
| | 1 = barely perceptible (fine) scales/flakes (mild) |
| | 2 = clearly perceptible (medium sized scales/flakes that are identifiable as individual |
| | scales/flakes on close inspection) scales/flakes OR prominent (thick, large scales/flakes |
| | that are easily identifiable as individual scales/flakes) scales/flakes over a limited area |
| | (moderate) |
| | 3 = prominent (thick, large scales/flakes that are easily identifiable as individual |
| | scales/flakes) scales/flakes over widespread areas(severe) |
| Lichenification^{b} | 0 = absent |
| | 1 = barely perceptible (Slight thickening of the skin with skin markings minimally |
| | exaggerated) thickening/exaggerated skin markings |
| | 2 = clearly perceptible (Definite thickening of the skin with skin markings clearly |
| | exaggerated) thickening/exaggerated skin markings OR prominent (Thickened |
| | indurated skin with skin markings visibly portraying an exaggerated criss-cross pattern) |
| | thickening/exaggerated skin markings over a limited area (moderate) |
| | 3 = prominent (thickened indurated skin with skin markings visibly portraying an |
| | exaggerated criss-cross pattern) thickening/exaggerated skin markings over widespread |
| | areas (severe) |
| Vesiculation/ | 0 = absent |
| Erosion | 1 = vesicles or erosions affecting up to 10% of the hand/foot (mild) |
| | 2 = vesicles or erosions affecting between 10-30% of the hand/foot (moderate) |
| | 3 = vesicles or erosions affecting more than 30% of the hand/foot (severe) |
| Edema | 0 = absent |
| | 1 = barely perceptible (Slight elevation of skin surface discernible only by palpation) |
| | dermal swelling (mild) |
| | 2 = clearly perceptible (Visible elevation of skin surface) dermal swelling OR prominent |
| | (Skin surface grossly swollen/elevated) dermal swelling over a limited area (moderate) |
| | 3 = prominent (Skin surface grossly swollen/elevated) dermal swelling over widespread |
| | areas (severe) |
| Fissures | 0 = absent |
| | 1 = barely perceptible (superficial) fissuring (mild) |
| | 2 = clearly perceptible (a single deep) fissuring OR prominent (multiple deep) fissuring |
| | over limited areas (moderate) |
| | 3 = prominent (multiple deep) fissuring over widespread areas (severe) |

^{a}Hyperkeratosis is not explicitly included as a separate sign, but hyperkeratosis as seen in hyperkeratotic eczema should be captured by scaling, fissures, and skin thickening
^{b}Palmar hyperlinearity as seen in ichthyosis vulgaris is not to be taken into account while assessing exaggerated skin markings

Investigator's Global Assessment of Hand and Foot: The IGA for hands and feet is an adaptation of the physician global assessment instrument which has been previously used in registrational studies in hand dermatitis (Ruzicka, et al., Br J Dermatol 2008; 158:808-17). Investigators assess the severity of signs of disease on hands and feet on this scale; this assessment is limited to only the hands and feet and is not influenced by severity of AD lesions on other parts of the body. The IGA score is assessed at screening, baseline, and on specified days during and/or after treatment, based on the scoring algorithm shown in Table 2 below. The assessment of the IGA should be based on overall impression of disease severity in hands and feet as per clinical judgement of investigator at the time of assessment. It is not necessary that all signs be present for a patient to be assigned to an IGA category.

TABLE 2
IGA for hand and foot

| Score | Category | IGA Morphological Descriptors |
|---|---|---|
| 0 | Clear | Absence of inflammatory signs of hand and/or foot atopic dermatitis |
| | | (erythema, scaling, lichenification, vesicles, fissures, edema). |
| 1 | Almost clear | Barely perceptible erythema, barely perceptible scaling, barely perceptible |
| | | lichenification and/or barely perceptible edema. No vesicles/erosions or |
| | | fissures. |
| 2 | Mild | Slight but definite erythema (pink), slight but definite scaling (fine scales), |
| | | slight but definite lichenification (slight thickening of the skin with skin |
| | | markings minimally exaggerated), slight but definite edema (elevation of skin |
| | | surface discernible only by palpation), slight but definite vesicles/erosions |
| | | (scattered, small vesicles or erosions) and/or slight but definite fissures (only |
| | | superficial fissures are present). |
| 3 | Moderate | Clearly perceptible erythema (dull red), clearly perceptible scaling |
| | | (moderately coarse scales), clearly perceptible lichenification (definite |
| | | thickening of the skin with skin markings clearly exaggerated), clearly |
| | | perceptible edema (visible elevation of skin surface), clearly perceptible |
| | | vesicles/erosions (larger vesicles or erosions) and/or clearly perceptible |
| | | fissures (predominantly superficial fissures but deep fissures may be present). |
| 4 | Severe | Prominent erythema (deep dark red), prominent scaling (thick, large scales), |
| | | prominent lichenification (thickened indurated skin with skin markings visibly |
| | | portraying an exaggerated criss-cross pattern or with deep furrows), |
| | | prominent edema (skin surface grossly swollen/elevated), prominent |
| | | vesicles/erosions (bullae, or densely, clustered vesicles or erosions, often with |
| | | oozing and crusting) and/or prominent fissures (predominantly deep fissures |
| | | are present). |

Notes:
1. In case of indeterminate cases:
(a) Use extent to guide rating of IGA severity for example, a patient with prominent erythema and clearly perceptible scaling, with limited extent of disease which would be classified as moderate IGA.
(b) Use severity rating for vesicles and fissures (signs with greater impact on patient's QoL) to step up rating of IGA severity. For example, a patient with clearly perceptible vesicles and slight but definite erythema should be assigned moderate IGA score.
2. Non-inflammatory signs of atopic dermatitis such as post-inflammatory hyper- or hypo-pigmentation, skin dryness etc. should not be used in assessment of IGA.

Hand and Foot Pruritus Numerical Rating Scale: The Pruritus NRS is a patient-reported assessment tool for evaluating the intensity of their hand and foot pruritus (itch) during a 24-hour recall period. This is an 11-point scale (0 to 10), in which 0 indicates no itching while 10 indicates worst itching possible. Patients complete the rating scale daily at specified time points during the study. The pruritus NRS score is calculated as the average of the last 7 days with a minimum of 4 daily scores.

Hand and Foot Skin Pain Numerical Rating Scale: Hand and foot skin pain is measured using a skin pain NRS. This is an 11-point scale (0 to 10) in which 0 indicates no pain while 10 indicates worst pain possible. Patients complete the rating scale daily at specified time points during the study.

Sleep Numerical Rating Scale: Sleep quality is measured using a sleep quality NRS. This is an 11-point scale (0 to 10) in which 0 indicates worst possible sleep while 10 indicates best possible sleep. The patient is asked to select the number that best describes the quality of their sleep during the previous night. Patients complete the rating scale daily at specified time points during the study.

Dermatology Life Quality Index: This questionnaire is based on general AD, not only on AD of the hand and/or foot. For adults, the DLQI is a 10-item, validated questionnaire used in clinical practice and clinical trials to assess the impact of AD disease symptoms and treatment on QOL (Badia, et al., Br J Dermatol, 1999, 141:698-702). The format is a simple response (0 to 3, where 0=“not at all”, 1=“only a little”; 2=“quite a lot”; and 3=“very much”) to 10 questions, which assess QOL over the past week, with an overall scoring system of 0 to 30; a high score is indicative of a poor QOL. The DLQI is assessed at specified time points during the study.

For adolescents, the CDLQI is a validated questionnaire designed to measure the impact of skin disease on the QOL in children (Lewis-Jones, et al., Br J Dermatol, 1995, 132:942-949). The aim of the questionnaire is to measure how much a patient's skin problem has affected the patient over a recall period of the past week. To complete the questionnaire, patients need to provide responses to 10 questions (the questions focus on domains such as symptoms feelings associated with disease, the impact of the disease on leisure, school or holidays, personal relationships, sleep, and side effects of treatment for the skin disease. The instrument has a recall period of 7 days. Nine of the 10 questions are scored as 0 to 3 (where 0=“not at all” or question unanswered, 1=“only a little”; 2=“quite a lot”; and 3=“very much”). Question 7 has an additional possible response (prevented school), which is assigned a score of 3. The CDLQI for a patient is the sum of the score of each question with a maximum of 30 and a minimum of 0. The higher the score, the greater the impact is on the QOL. The CDLQI is assessed at specified time points during the study.

Hospital Anxiety and Depression Scale: This questionnaire is based on general AD, not only on AD of the hand and/or foot. The HADS is an instrument for screening anxiety and depression in non-psychiatric populations; repeated administration also provides information about changes to a patient's emotional state (Zigmond and Snaith, 1983, Acta Psychiatr. Scand, 67: 361-70; Herrmann, 1997, J. Psychosom. Res., 42: 17-41). The HADS consists of 14 items, 7 each for anxiety and depression symptoms; possible scores range from 0 to 21 for each subscale. The following cut-off scores are recommended for both subscales: 7 to 8 for possible presence, 10 to 11 for probable presence, and 14 to 15 for severe anxiety or depression. The questionnaire is administered to patients at specified time points during the study.

Patient Oriented Eczema Measure: The POEM is a 7-item, validated questionnaire used in clinical practice and clinical trials to assess disease symptoms in children and adults (Charman, et al., Archives of Dermatology, 2004, 140:1513-1519). The format is a response to 7 items (dryness, itching, flaking, cracking, sleep loss, bleeding, and weeping) based on frequency of these disease symptoms during the past week (i.e., 0=no days, 1=1 to 2 days, 2=3 to 4 days, 3=5 to 6 days, and 4=all days) with a scoring system of 0 to 28; the total score reflects disease-related morbidity. The POEM questionnaire is assessed at specified time points during the study.

Hand Eczema Severity Index: The HECSI is similar to scoring systems in AD (EASI) and Psoriasis vulgaris (PASI) in incorporating both the extent and the intensity of the disease. Each hand is divided into 5 areas [fingertips, fingers (except the tips), palms, back of hands and wrists]. For each of these areas the intensity of the 6 following clinical signs: erythema, induration/papulation, vesicles, fissuring, scaling and oedema is graded on the following scale: 0, no skin changes; 1, mild disease; 2, moderate, and 3, severe. For each location (total of both hands) the affected area is given a score from 0 to 4 (0, 0%; 1, 1 to 25%; 2, 26 to 50%; 3, 51 to 75%, and 4, 76 to 100%) for the extent of clinical symptoms. Finally, the score given for the extent at each location is multiplied by the total sum of the intensity of each clinical feature, and the total sum called the HECSI score is calculated, varying from 0 to a maximum severity score of 360 points. The HECSI has been previously validated in patients with hand dermatitis (Held, et al., Br J Dermatol, 2005, 152:302-307).

Quality of Life in Hand Eczema Questionnaire: The QoLHEQ is a disease-specific instrument to assess health-related Quality of Life (HRQOL) in hand eczema (HE) patients (Ofenloch, et al., Br J Dermatol, 2014, 171: 304-312. The QOLHEQ consists of 30 items assessing four domains of HRQOL: (a) symptoms, (b) emotions, (c) functioning and (d) treatment/prevention. The QOLHEQ total-score ranges from 0-127 points. The QoLHEQ is assessed at specified time points during the study.

### Results

A total of 133 patients were enrolled in an approximately 1:1 randomization of dupilumab (n=67) every two weeks (adults 300 mg, adolescents 200 mg or 300 mg based on body weight) or placebo (n=66). Of these patients, a higher percentage of patients in the dupilumab group completed treatment as compared to placebo (94.0% for dupilumab; 81.5% for placebo; 88.0% total patients).

Baseline demographics and disease characteristics are summarized in Tables 3 and 4. Baseline demographics were generally balanced between the treatment arms, with a slight predilection towards female patients consistent with background rates reported in the literature (Table 3). As shown in Table 4, disease severity was balanced across the treatment arms. Trial participants had substantially chronic disease, with the majority having disease on both hands and feet. The study population had a high baseline disease severity as reflected by measures of signs, symptoms, and quality of life. Approximately two-thirds of patients either had disease localized to hands and feet or had mild disease (EASI <16). A significant number of patients had prior use of systemic immunosuppressants, reflecting the high patient burden imposed by the disease on hands and feet.

TABLE 3
Baseline Demographics

| | Placebo | | Dupilumab | | Total | |
|---|---|---|---|---|---|---|
| n (Full Analysis | 66 | | 67 | | 133 | |
| Set (FAS)) | | | | | | |
| Age (years), | 33.4 | (14.66) | 35.8 | (17.07) | 34.6 | (15.90) |
| mean (SD) | | | | | | |
| ≥12 yr to <18 | 13 | (19.7%) | 14 | (20.9%) | 27 | (20.3%) |
| yr, n (%) | | | | | | |
| ≥18 yr, n (%) | 53 | (80.3%) | 53 | (79.1%) | 106 | (79.7%) |
| Gender, n (%) | | | | | | |
| Male | 28 | (42.4%) | 22 | (32.8%) | 50 | (37.6%) |
| Female | 38 | (57.6%) | 45 | (67.2%) | 83 | (62.4%) |
| Race, n (%) | | | | | | |
| White | 53 | (80.3%) | 53 | (79.1%) | 106 | (79.7%) |
| Black or African | 4 | (6.1%) | 3 | (4.5%) | 7 | (5.3%) |
| American | | | | | | |
| Asian | 8 | (12.1%) | 9 | (13.4%) | 17 | (12.8%) |
| Other | 1 | (1.5%) | 2 | (3.0%) | 3 | (2.3%) |

TABLE 4
Baseline Disease Characteristics

| | Placebo | | Dupilumab | | Total | |
|---|---|---|---|---|---|---|
| n (FAS) | 66 | | 67 | | 133 | |
| Duration of hand and/or foot AD | 15.4 | (13.18) | 15.7 | (12.84) | 15.6 | (12.96) |
| (yrs), mean (SD) | | | | | | |
| Age at onset of hand and/or foot | 17.8 | (17.09) | 20.1 | (19.05) | 18.9 | (18.07) |
| AD (yrs), mean (SD) | | | | | | |
| Morphology of hand and/or foot AD | | | | | | |
| Chronic dry fissured | 26 | (39.4%) | 37 | (55.2%) | 63 | (47.4%) |
| Hyperkeratotic | 19 | (28.8%) | 18 | (26.9%) | 37 | (27.8%) |
| Dyshidrotic | 8 | (12.1%) | 5 | (7.5%) | 13 | (9.8%) |
| Other¹ | 13 | (19.7%) | 7 | (10.4%) | 20 | (15.0%) |
| Number of patients with hand and | | | | | | |
| foot involvement, n (%) | | | | | | |
| Both hand and foot | 36 | (54.5%) | 35 | (52.2%) | 71 | (53.4%) |
| Foot only | 1 | (1.5%) | 3 | (4.5%) | 4 | (3.0%) |
| Hand only | 29 | (43.9%) | 29 | (43.3%) | 58 | (43.6%) |
| Number of patients with positive patch | | | | | | |
| test to one or more allergens, n (%)² | | | | | | |
| Yes | 14 | (21.5%) | 17 | (26.2%) | 31 | (23.8%) |
| No | 51 | (78.5%) | 48 | (73.8%) | 99 | (76.2%) |
| IGA of hand and foot score (0-4), n (%) | | | | | | |
| Moderate (IGA = 3) | 48 | (72.7%) | 48 | (71.6%) | 96 | (72.2%) |
| Severe (IGA = 4) | 18 | (27.3%) | 19 | (28.4%) | 37 | (27.8%) |
| mTLSS for hand and foot (0-36), mean (SD) | 16.0 | (5.76) | 17.0 | (5.95) | 16.5 | (5.86) |
| Hand and Foot Peak Pruritus NRS (0-10), | 6.9 | (1.45) | 7.2 | (1.50) | 7.1 | (1.48) |
| mean (SD) | | | | | | |
| Hand and Foot Skin Peak Pain NRS (0-10), | 6.0 | (2.46) | 6.8 | (2.02) | 6.4 | (2.27) |
| mean (SD) | | | | | | |
| Sleep NRS (0-10), mean (SD) | 5.4 | (1.88) | 5.5 | (1.89) | 5.4 | (1.88) |
| Hand and Foot Area Involvement of Atopic | 27.4 | (15.65) | 24.6 | (15.20) | 26.0 | (15.43) |
| Dermatitis (0-100), mean (SD) | | | | | | |
| HECSI for patients with hand eczema | 47.4 | (18.48) | 46.2 | (14.38) | 46.8 | (16.50) |
| (0-360), mean (SD) | | | | | | |
| QOLHEQ for patients with hand eczema | 70.8 | (23.94) | 74.3 | (23.93) | 72.5 | (23.90) |
| (0-117), mean (SD) | | | | | | |
| Number of patients with AD outside hands | 50 | (75.7%) | 46 | (68.7%) | 96 | (72.2%) |
| and feet (%) | | | | | | |
| EASI for patients with AD outside hands | | | | | | |
| and feet | | | | | | |
| Mean (SD) | 17.0 | (13.90) | 20.5 | (13.87) | 18.7 | (13.92) |
| <16 | 26 | (39.4%) | 20 | (29.9%) | 46 | (34.6) |
| ≥16 | 24 | (36.4%) | 26 | (38.8%) | 50 | (37.6%) |
| IGA for patients with AD outside hands | | | | | | |
| and feet | | | | | | |
| Almost clear | 5 | (10.0%) | 2 | (4.3%) | | |
| Mild | 10 | (20.0%) | 9 | (19.6%) | | |
| Moderate | 28 | (56.0%) | 30 | (65.2%) | | |
| Severe | 7 | (14.0%) | 5 | (10.9%) | | |
| Prior systemic medications for AD, n (%) | 16 | (24.2%) | 25 | (37.3%) | 41 | (30.8%) |
| Prior systemic corticosteroids, n (%) | 9 | (13.6%) | 11 | (16.4%) | 20 | (15.0%) |
| Prior systemic non-steroidal | 8 | (12.1%) | 17 | (25.4%) | 25 | (18.8%) |
| immunosuppressants, n (%) | | | | | | |

¹Other included pulpitis, nummular eczema, unspecified and patient specified predominant morphology
²Patients who had patch test done within last 3 years prior to screening did not require patch test to be performed at screening

As shown in Table 5, there was a high incidence of atopic co-morbidities in the patient population, underlining the common Type 2 pathophysiology behind these diseases.

TABLE 5
Concurrent Atopic/Allergic Conditions

| | Placebo | Dupilumab | Total |
|---|---|---|---|
| n (FAS) | 66 | 67 | 133 |
| Number (%) of patients | 42 (63.6%) | 49 (73.1%) | 91 (68.4%) |
| with current history | | | |
| of atopic/allergic | | | |
| conditions excluding | | | |
| atopic dermatitis | | | |
| Allergic Rhinitis | 26 (39.4%) | 34 (50.7%) | 60 (45.1%) |
| Asthma | 14 (21.2%) | 25 (37.3%) | 39 (29.3%) |
| Food Allergy | 11 (16.7%) | 11 (16.4%) | 39 (29.3%) |
| Allergic Conjunctivitis | 11 (16.7%) | 17 (25.4%) | 28 (21.1%) |
| Hives | 3 (4.5%) | 1 (1.5%) | 4 (3.0%) |
| Chronic Rhinosinusitis | 2 (2.6%) | 1 (1.2%) | 3 (1.9%) |
| Nasal Polyps | 2 (2.6%) | 1 (1.2%) | 3 (1.9%) |
| Other Allergies* | 14 (21.2%) | 21 (31.3%) | 35 (26.3%) |

*Refers to allergies to plants, animals, dust mite, medications, etc.

### Efficacy

Treatment with dupilumab met all prespecified efficacy endpoints with highly significant p-values. See Table 6. The primary endpoint evaluated the proportion of patients with clear or almost clear skin of the hands and feet at 16 weeks (measured by a score of 0 or 1 on the Investigator's Global Assessment (IGA) for hand and foot). At Week 16, 40.3% of dupilumab-treated patients achieved an IGA for hand and foot of 0/1, compared to only 16.7% of placebo-treated patients. A statistically significant improvement in the primary endpoint was apparent at Week 4 and sustained through Week 16.

The key secondary endpoint measured the proportion of patients with improvement in itch from baseline (measured by a ≥4-point reduction in Peak-Pruritis Numeric Rating Scale [PP-NRS] on a 0-10 scale) at 16 weeks. The onset of improvement in pruritus of hands and feet was rapid (by Week 1) with dupilumab treatment, and was sustained until Week 16. At Week 16, 52.2% of dupilumab-treated patients achieved a ≥4-point reduction in PP-NRS, compared to only 13.6% of placebo-treated patients.

To evaluate the effect of dupilumab treatment on individual signs of atopic hand and/or foot dermatitis, the proportion of patients reporting absent, mild, moderate, or severe erythema, scaling/flaking, lichenification, vesiculation/erosion, edema, and fissures, as assessed by mTLSS in hands and feet, was analyzed. Of the 133 patients enrolled, over 65% of patients treated with dupilumab (n=67) achieved an absent or mild score by Week 16 in each of the signs/symptoms assessed. Proportion of patients with absent or mild hand scores increased from baseline to Week 16 in erythema (9% vs 71.6%), scaling/flaking (16.4% vs 74.7%), lichenification (4.5% vs 65.6%), vesiculation/erosion (43.3% vs 89.6%), edema (44.7% vs 86.6%), and fissures (23.9% vs 83.5%). The proportion of patients with absent or mild foot scores increased from baseline to Week 16 in erythema (56.7% vs 80.6%), scaling/flaking (56.7% vs 82.1%), lichenification (53.8% vs 82.1%), vesiculation/erosion (76.1% vs 86.6%), edema (76.1% vs 88.1%), and fissures (77.6% vs 86.6%).

TABLE 6
Efficacy Results for Primary and Secondary Endpoints

| | | Placebo | | Dupilumab | | Change vs. placebo | | P-value vs. |
|---|---|---|---|---|---|---|---|---|
| Level | Week 16 Endpoints | (n = 66) | | (n = 67) | | (95% CI) | | placebo |
| Primary | Proportion of patients with IGA (hand and foot) 0 to 1, | 11 | (16.7) | 27 | (40.3) | 23.6 | (8.84, 38.42) | 0.0030 |
| | n (%) [score range 0-4] | | | | | | | |
| Key | Proportion of patients with improvement of hand and | 9 | (13.6) | 35 | (52.2) | 38.6 | (24.06, 53.15) | <0.0001 |
| Secondary | foot pruritus NRS ≥4, n (%) [score range 0-10] | | | | | | | |
| Other | Percent change from baseline in mTLSS for | −29.1 | (5.88) | −67.4 | (5.90) | −38.3 | (−50.23, −26.28) | <0.0001 |
| Secondary | hand/foot lesions, LS Mean (SE) [score range 0-36] | | | | | | | |
| | Percent change from baseline in weekly average of | −24.3 | (5.94) | −61.5 | (5.90) | −37.2 | (−49.20, −25.21) | <0.0001 |
| | daily hand and foot peak pruritus NRS, LS Mean (SE) | | | | | | | |
| | Change from baseline in weekly average of daily | −1.8 | (0.43) | −4.6 | (0.43) | −2.8 | (−3.68, −1.90) | <0.0001 |
| | hand and foot peak Pain NRS, LS Mean (SE) [score | | | | | | | |
| | range 0-10] | | | | | | | |
| | Percent change from baseline in Hand Eczema | −37.7 | (6.31) | −73.4 | (6.37) | −35.7 | (−48.45, −22.93) | <0.0001 |
| | Severity Index (HECSI) score, LS Mean (SE) [score | | | | | | | |
| | range 0-360] | | | | | | | |
| | Proportion of patients with HECSI-75, n (%) | 14 | (21.5) | 30 | (46.9) | 25.3 | (9.55, 41.13) | 0.0028 |
| | Change from baseline in percent surface area of | −9.1 | (2.54) | −15.7 | (2.51) | −6.6 | (−11.70, −1.45) | 0.0119 |
| | hand and foot involvement with AD, LS Mean (SE) | | | | | | | |
| | [score range 0-100] | | | | | | | |
| | Change from baseline in QoLHEQ, LS Mean (SE) | −14.9 | (4.12) | −40.1 | (4.01) | −25.2 | (−33.24, −17.12) | <0.0001 |
| | [score range 0-117] | | | | | | | |
| | Change from baseline in weekly average of daily | −0.02 | (0.33) | 0.9 | (0.33) | 0.9 | (0.22, 1.56) | 0.0090 |
| | Sleep NRS, LS Mean (SE) [score range 0-10] | | | | | | | |

### Use of Rescue Medication

The use of rescue medication was seven times higher in placebo-treated patients as compared to dupilumab-treated patients (14/66 (21.2%) for placebo vs. 2/67 (3.0%) for dupilumab at Week 16). See Table 7 below. No patients in the dupilumab-treated group required rescue with systemic medication.

TABLE 7
Rescue Medication Taken During 16-Week Treatment Period

| | Placebo | | Dupilumab | Total | |
|---|---|---|---|---|---|
| | (n = 66) | | (n = 67) | (n = 133) | |
| Patients with at least one | 14 | (21.2%) | 2 (3.0%) | 16 | (12.0%) |
| rescue medication, n (%) | | | | | |
| Corticosteroids, | 13 | (19.7%) | 2 (3.0%) | 15 | (11.3%) |
| dermatological preparations | | | | | |
| Corticosteroids, moderately | 1 | (1.5%) | 0 | 1 | (0.8%) |
| potent (Group II) | | | | | |
| Corticosteroids, | 5 | (7.6%) | 1 (1.5%) | 6 | (4.5%) |
| potent (Group III) | | | | | |
| Corticosteroids, very | 9 | (13.6%) | 1 (1.5%) | 10 | (7.5%) |
| potent (Group IV) | | | | | |
| Corticosteroids, potent, | 2 | (3.0%) | 0 | 2 | (1.5%) |
| combinations with antiseptics | | | | | |
| Corticosteroids for | 1 | (1.5%) | 0 | 1 | (0.8%) |
| systemic use | | | | | |
| Glucocorticoids | 1 | (1.5%) | 0 | 1 | (0.8%) |
| Immunosuppressants | 1 | (1.5%) | 0 | 1 | (0.8%) |
| Calcineurin inhibitors | 1 | (1.5%) | 0 | 1 | (0.8%) |

### Safety

Dupilumab was well tolerated and demonstrated an acceptable safety profile, with no new safety concerns identified. See Table 8. For the 16-week treatment period, overall rates of treatment-emergent adverse events (TEAEs) were 65.7% for dupilumab and 74.2% for placebo. There was a low incidence of serious adverse events (SAEs) and AEs leading to permanent treatment discontinuation. A higher incidence of conjunctivitis and a lower incidence of COVID-19 infections were seen in the dupilumab arm.

TABLE 8
Treatment-Emergent Adverse Events

| | Placebo | Dupilumab | |
|---|---|---|---|
| | % Patients | | |
| | n = 66 | n = 67 | |
| Deaths | 0 | 0 | |
| Any TEAE | 49 (74.2%) | 44 | (65.7%) |
| Any drug related TEAE | 5 (7.6%) | 8 | (11.9%) |
| Any TE SAE | 1 (1.5%) | 2 | (3.0%) |
| Any drug related TE SAE | 0 | 0 | |
| Any TEAE leading to discontinuation of | 3 (4.5%) | 1 | (1.5%) |
| study drug permanently | | | |
| TEAE of Special Interest (Adjudicated) | 0 | 0 | |
| Conjunctivitis (Narrow CMQ) | 1 (1.5%) | 4 | (6.0%) |
| Conjunctivitis (Broad CMQ) | 1 (1.5%) | 7 | (10.4%) |
| Skin infection of hands and feet | 1 (1.5%) | 0 | |
| excluding herpes infections (Adjudicated) | | | |
| Skin infections overall excluding herpes | 1 (1.5%) | 1 | (1.5%) |
| infections (Adjudicated) | | | |
| Injection site reactions (HLT) | 2 (3.0%) | 4 | (6.0%) |
| Herpes viral infections of hand and feet | 0 | 1 | (1.5%) |
| (HLT) | | | |
| Herpes viral infections overall (HLT) | 2 (3.0%) | 4 | (6.0%) |
| COVID-19 (Narrow SMQ) | 6 (9.1%) | 2 | (3.0%) |

### SUMMARY

This Phase 3 trial evaluated the efficacy and safety of dupilumab in 133 adolescents and adults with moderate-to-severe atopic dermatitis of the hands and feet who had an inadequate response or intolerance to topical corticosteroids. Patients with irritant contact dermatitis were excluded from the trial. Atopic and allergic comorbidities were present in 73% and 64% of dupilumab-treated and placebo-treated patients, respectively.

Patients received dupilumab (n=67) every two weeks (adults 300 mg, adolescents 200 mg or 300 mg based on body weight) or placebo (n=66). At 16 weeks, among patients treated with dupilumab, more than twice as many achieved clear or almost clear skin (40% compared to 17% with placebo (p=0.0030)). For the key secondary endpoint, 52% of dupilumab-treated experienced a clinically meaningful reduction in itch compared to 14% with placebo (p<0.0001). There was a 73% average improvement in disease severity from baseline for dupilumab-treated patients, compared to 38% with placebo (p<0.0001). Dupilumab-treated patients also exhibited significant improvements in measures of skin pain, sleep and health-related quality of life as compared to placebo-treated patients. Dupilumab rapidly improved disease signs and symptoms, significantly reducing itch as early as one week, and improving pain and health-related quality of life in two weeks. Furthermore, the required use of rescue medications was significantly lower for dupilumab-treated patients as compared to placebo (3% versus 21%).

The trial demonstrated similar safety results to the known safety profile of dupilumab in atopic dermatitis. For the 16-week treatment period, overall rates of adverse events (AEs) were 66% for dupilumab and 74% for placebo. AEs more commonly observed with dupilumab (5%) included conjunctivitis (6% dupilumab, 2% placebo) and herpes viral infections (6% dupilumab, 3% placebo).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 9
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| 7 | LGS | Dupilumab LCDR2 amino |
| | | acid sequence |
| 8 | MQALQTPYT | Dupilumab LCDR3 amino |
| | | acid sequence |
| 9 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27-6-33-VH |
| | TYYANSVKGRFTISRDNPKNTLFLQMSSLRAEDTAVYYCVRSKVRYRGGMDVWGQ | |
| | GTTVTVSS | |
| 210 | EVQLLESGGGLVQPGGSLRLSCAASGFTLSSYAMHWVRQAPGKGLEYVSGISPSGVS | STSA-C27-7-33-VH |
| | TYYANSVKGRFTISRDNPKNTLFLQMSSLRAEDTAVYYCVRVKVKYRGGMDVWGQ | |
| | GTTVTVSS | |
| 211 | EVQLLESGGGLVQPGGSLRLSCAASGFTLSSYAMHWVRQAPGKGLEYVSGISPTSGS | STSA-C27-24-56-VH |
| | TYYANSVKGRFTISRDNPKNTLFLQMSSLRAEDTAVYYCVRVKVRYRGGMDVWGQ | |
| | GTTVTVSS | |
| 212 | EVQLLESGGGLVQPGGSLRLSCAASGFTLSSYAMHWVRQAPGKGLEYVSGISPTGTS | STSA-C27-47-56-VH |
| | TYYANSVKGRFTISRDNPKNTLFLQMSSLRAEDTAVYYCVRVKGAYRGGMDVWGQ | |
| | GTTVTVSS | |
| 213 | EVQLLESGGGLVQPGGSLRLSCAASGFTLSSYAMHWVRQAPGKGLEYVSGISSSGSS | STSA-C27-33-33-VH |
| | TYYANSVKGRFTISRDNPKNTLFLQMSSLRAEDTAVYYCVRVKVAYRGGMDVWGQ | |
| | GTTVTVSS | |
| 214 | EVQLLESGGGLVQPGGSLRLSCAASGFTLSSYAMHWVRQAPGKGLEYVSGISPSSTS | STSA-C27-56-56-VH |
| | TYYANSVKGRFTISRDNPKNTLFLQMSSLRAEDTAVYYCVRVKVLYRGGMDVWGQ | |
| | GTTVTVSS | |
| 215 | EVQLLESGGGLVQPGGSLRLSCAASGFTLSSYAMHWVRQAPGKGLEYVSGISPSSAS | STSA-C27-78-78-VH |
| | TYYANSVKGRFTISRDNPKNTLFLQMSSLRAEDTAVYYCVRVKSKYRGGMDVWGQ | |
| | GTTVTVSS | |
| 216 | EVQLLESGGGLVQPGGSLRLSCAASGFTLSSYAMHWVRQAPGKGLEYVSGISGNSAS | STSA-C27-82-58-VH |
| | TYYANSVKGRFTISRDNPKNTLFLQMSSLRAEDTAVYYCVRVKLKYRGGMDVWGQG | |
| | TTVTVSS | |
| 217 | EVQLLESGGGLVQPGGSLRLSCAASGFTLSSYAMHWVRQAPGKGLEYVSGISHSGTS | STSA-C27-54-54-VH |
| | TYYANSVKGRFTISRDNPKNTLFLQMSSLRAEDTAVYYCVRVRVLYRGGMDVWGQ | |
| | GTTVTVSS | |
| 218 | EVQLLESGGGLVQPGGSLRLSCAASGFTLSSYAMHWVRQAPGKGLEYVSGISPSGVS | STSA-C27-36-36-VH |
| | TYYANSVKGRFTISRDNPKNTLFLQMSSLRAEDTAVYYCVRVKVKYRGGMDVWGQ | |
| | GTTVTVSS | |
| 219 | EVQLLESGGGLVQPGGSLRLSCAASGFTLSSYAMHWVRQAPGKGLEYVSGISSNGG | STSA-C27-53-53-VH |
| | STYYANSVKGRFTISRDNPKNTLFLQMSSLRAEDTAVYYCVRVFVRYRGGMDVWGQ | |
| | GTTVTVSS | |
| 220 | EVQLLESGGGLVQPGGSLRLSCAASGFTLSSYAMHWVRQAPGKGLEYVSGISPTSAS | STSA-C27-67-67-VH |
| | TYYANSVKGRFTISRDNPKNTLFLQMSSLRAEDTAVYYCVRVKGRYRGGMDVWGQ | |
| | GTTVTVSS | |
| 221 | EVQLLESGGGLVQPGGSLRLSCAASGFTLSSYAMHWVRQAPGKGLEYVSGISPTGGS | STSA-C27-55-55-VH |
| | TYYANSVKGRFTISRDNPKNTLFLQMSSLRAEDTAVYYCVRVKGRYRGGMDVWGQ | |
| | GTTVTVSS | |
| 222 | EVQLLESGGGLVQPGGSLRLSCAASGFTLSSYAMHWVRQAPGKGLEYVSGISHSGN | STSA-C27-59-59-VH |
| | STYYANSVKGRFTISRDNPKNTLFLQMSSLRAEDTAVYYCVRVKRRYRGGMDVWGQ | |
| | GTTVTVSS | |
| 223 | EVQLLESGGGLVQPGGSLRLSCAASGFTLSSYAMHWVRQAPGKGLEYVSGISPSSNS | STSA-C27-58-58-VH |
| | TYYANSVKGRFTISRDNPKNTLFLQMSSLRAEDTAVYYCVRVKVRYRGGMDVWGQ | |
| | GTTVTVSS | |
| 224 | EVQLLESGGGLVQPGGSLRLSCAASGFTLSSYAMHWVRQAPGKGLEYVSGISSSGSS | STSA-C27-52-52-VH |
| | TYYANSVKGRFTISRDNPKNTLFLQMSSLRAEDTAVYYCVRVKPAYRGGMDVWGQ | |
| | GTTVTVSS | |
| 225 | EVQLLESGGGLVQPGGSLRLSCAASGFTLSSYAMHWVRQAPGKGLEYVSGISYSSAS | STSA-C27-Y2-Y2-VH |
| | TYYANSVKGRFTISRDNPKNTLFLQMSSLRAEDTAVYYCVRVKVRYRGGMDVWGQ | |
| | GTTVTVSS | |
| 226 | ETTLTQSPDTLPLSPGDRASLSCRASQSVSSAYLAWYQQKPGQAPRLLIYGTSRRATG | STSA-C27-VL |
| | VPGRFSGSGSGTDFTLTISRLEPEDFAVYYCQLYGSSSVTFGQGTKLEIK | |
| 227 | EIVLTQSPGTLSLSPGERATLSCRASQGISSAYLAWYQQKPGQAPRLLIYGTSRRATGI | STSA-C27-6-33-VL |
| | PDRFSGSGSGTDFTLTISRLEPEDFAVYYCQLYGATSVTFGQGTKLEIK | |
| 228 | EIVLTQSPGTLSLSPGERATLSCRASQGISSAYLAWYQQKPGQAPRLLIYGTSRRATGI | STSA-C27-7-33-VL |
| | PDRFSGSGSGTDFTLTISRLEPEDFAVYYCQLYGATSVTFGQGTKLEIK | |
| 229 | EIVLTQSPGTLSLSPGERATLSCRASQSVSSAYLAWYQQKPGQAPRLLIYGTSRRATGI | STSA-C27-24-56-VL |
| | PDRFSGSGSGTDFTLTISRLEPEDFAVYYCQLYGASSVTFGQGTKLEIK | |
| 230 | EIVLTQSPGTLSLSPGERATLSCRASQSVSSAYLAWYQQKPGQAPRLLIYGTSRRATGI | STSA-C27-47-56-VL |
| | PDRFSGSGSGTDFTLTISRLEPEDFAVYYCQLYGASSVTFGQGTKLEIK | |
| 231 | EIVLTQSPGTLSLSPGERATLSCRASQGISSAYLAWYQQKPGQAPRLLIYGTSRRATGI | STSA-C27-33-33-VL |
| | PDRFSGSGSGTDFTLTISRLEPEDFAVYYCQLYGATSVTFGQGTKLEIK | |
| 232 | EIVLTQSPGTLSLSPGERATLSCRASQSVSSAYLAWYQQKPGQAPRLLIYGTSRRATGI | STSA-C27-56-56-VL |
| | PDRFSGSGSGTDFTLTISRLEPEDFAVYYCQLYGASSVTFGQGTKLEIK | |
| 233 | EIVLTQSPGTLSLSPGERATLSCRASQSISTAYLAWYQQKPGQAPRLLIYGTSRRATGIP | STSA-C27-78-78-VL |
| | DRFSGSGSGTDFTLTISRLEPEDFAVYYCQLYGASSVTFGQGTKLEIK | |
| 234 | EIVLTQSPGTLSLSPGERATLSCRASQDISSAYLAWYQQKPGQAPRLLIYGTSRRATGI | STSA-C27-82-58-VL |
| | PDRFSGSGSGTDFTLTISRLEPEDFAVYYCQLYGATSVTFGQGTKLEIK | |
| 235 | EIVLTQSPGTLSLSPGERATLSCRASQDVSSAYLAWYQQKPGQAPRLLIYGTSRRATGI | STSA-C27-54-54-VL |
| | PDRFSGSGSGTDFTLTISRLEPEDFAVYYCQLYGATSVTFGQGTKLEIK | |
| 236 | EIVLTQSPGTLSLSPGERATLSCRASQNISTAYLAWYQQKPGQAPRLLIYGTSRRATGI | STSA-C27-36-36-VL |
| | PDRFSGSGSGTDFTLTISRLEPEDFAVYYCQLYGATSVTFGQGTKLEIK | |
| 237 | EIVLTQSPGTLSLSPGERATLSCRASQDASNAYLAWYQQKPGQAPRLLIYGTSRRATG | STSA-C27-53-53-VL |
| | IPDRFSGSGSGTDFTLTISRLEPEDFAVYYCQLYGSSSVTFGQGTKLEIK | |
| 238 | EIVLTQSPGTLSLSPGERATLSCRASQGVSSAYLAWYQQKPGQAPRLLIYGTSRRATGI | STSA-C27-67-67-VL |
| | PDRFSGSGSGTDFTLTISRLEPEDFAVYYCQLYGRSSVTFGQGTKLEIK | |
| 239 | EIVLTQSPGTLSLSPGERATLSCRASQNISTAYLAWYQQKPGQAPRLLIYGTSRRATGI | STSA-C27-55-55-VL |
| | PDRFSGSGSGTDFTLTISRLEPEDFAVYYCQLYGTSSVTFGQGTKLEIK | |
| 240 | EIVLTQSPGTLSLSPGERATLSCRASQSVSTAYLAWYQQKPGQAPRLLIYGTSRRATGI | STSA-C27-59-59-VL |
| | PDRFSGSGSGTDFTLTISRLEPEDFAVYYCQLYGATSVTFGQGTKLEIK | |
| 241 | EIVLTQSPGTLSLSPGERATLSCRASQDISSAYLAWYQQKPGQAPRLLIYGTSRRATGI | STSA-C27-58-58-VL |
| | PDRFSGSGSGTDFTLTISRLEPEDFAVYYCQLYGATSVTFGQGTKLEIK | |
| 242 | EIVLTQSPGTLSLSPGERATLSCRASQGVSTAYLAWYQQKPGQAPRLLIYGTSRRATGI | STSA-C27-52-52-VL |
| | PDRFSGSGSGTDFTLTISRLEPEDFAVYYCQLYGATSVTFGQGTKLEIK | |
| 243 | EIVLPQSPGTLSLSPGERATLSCRASQGVSSAYLAWYQQKPGQAPRLLIYGTSRRATGI | STSA-C27-Y2-Y2-VL |
| | PDRFSGSGSGTDFTLTISRLEPEDFAVYYCQLYGSTSVTFGQGTKLEIK | |
| 244 | EVQLVESGGGLVQPKGSLKLSCAASGFTFNTYGMHWVRQAPGKGLEWVAHIRSKS | Y0188-1 VH |
| | SNYATYYADSVKDRFTISRDDSQSMLYLQMNNLKTEDTAMYYCVRWFRAMDYWG | |
| | QGTSVTVSS | |
| 245 | EVQLIESGGGLVQPKGSLKLSCAASGFTFNMYAMDWVRQAPGKGLEWVARIRSKG | Y0188-2 VH |
| | SNFETNYADSVKDRFTISRDDSQSMVYLQMINLKTEDTAMYYCVRHRGGAWFAYW | |
| | GQGTLVSVSA | |
| 246 | QVQLVETGGGLVRPGNSLKLSCVTSGFTFSNYRMHWLRQPPGKRLEWIAVITVKSN | Y0188-3 VH |
| | NYGANYAESVKGRFAISRDDSKSSVYLEMNRLREEDTATYFCSRERAYGNPFDYWG | |
| | QGTTLTVSS | |
| 247 | EVQLVESGGGLVQPKGSLKLSCAASGFTFNMYAMNWVRQAPGQGLEWVARIRSKS | Y0188-4 VH |
| | NNYATYYADSVKDRFIISRDDSESMVYLQMSNLRAADTAMYYCVRHLRAMDYWG | |
| | QGTSVTVSS | |
| 248 | EVQLVESGGGLVQPKGSLKLSCAASGFSFNMYAMNWVRQAPGKGLEWVARIRTKS | Y0188-6 VH |
| | NHYSTYYADSVKDRFTISRDDSASMFYLQMNNLKTEDTAMYFCVRHLRAMDYWG | |
| | QGTSVTVSS | |
| 249 | EVQLIESGGGLVQPKGSLKLSCAASGFTFNMYAMDWVRQAPGKGLEWVARIRSKG | Y0188-8 VH |
| | SNFETNYADSVKDRFTISRDDSQSMVYLQMNNLKTEDTAMYYCVRHRGGAWFAY | |
| | WGQGTLVTVSA | |
| 250 | EVQLVESGGGLVRPKGSLKLSCAASGFSFNTYAMNWVRQAPGKGLEWIVWIRSKSH | Y0188-9 VH |
| | NYATYYADSVKDRFTISRDDSESMLYLQMNNLKTEDTAMYYCVRHLRAMDYWGQ | |
| | GTSVTVSS | |
| 251 | EVRLVESGGGLVQPKGSLKLSCEASGFSFNMYAMNWVRQAPGKGLEWITHIRSKSN | Y0188-10 VH |
| | NYATYYADSVKDRFIISRDDSESMVYLQMNNLKTEDTAMYYCVRLLRALDYWGQGT | |
| | SVTVSS | |
| 252 | EVQLVESGGGLVQPKGSLKLSCAASGFTFNMYGMHWVRQAPGKGLEWVAHIRSKS | Y0188-14 VH |
| | SNYATYYADSVKDRLTISRDDSQSMLYLQMNNLKTEDTAMYYCVRWFRAMDYWG | |
| | QGTSVTVSS | |
| 253 | EVQLVESGGGLVKPGGSLRLSCAASGFTFSMYGMHWVRQAPGKGLEWVGHIRSKS | HV3-15-14 VH |
| | SNYATYYADSVKDRFTISRDDSKNTLYLQMNSLKTEDTAVYYCTTWFRAMDYWGQ | |
| | GTLVTVSS | |
| 254 | EVQLVESGGGLVQPGGSLRLSCAASGFTFSMYGMHWVRQAPGKGLEWVSHIRSKS | HV3-48-14 VH |
| | SNYATYYADSVKDRFTISRDNAKNSLYLQMNSLRAEDTAVYYCARWFRAMDYWGQ | |
| | GTLVTVSS | |
| 255 | EVQLVESGGGLVQPGGSLKLSCAASGFTFSMYGMHWVRQASGKGLEWVGHIRSKS | HV3-73*2-14 VH |
| | SNYATYYADSVKDRFTISRDDSKNTAYLQMNSLKTEDTAVYYCTRWFRAMDYWGQ | |
| | GTLVTVSS | |
| 256 | EVQLVESGGGLVQPGGSLRLSCAASGFTFSMYGMHWVRQAPGKGLEWVGHIRSKS | HV3-72-14 VH |
| | SNYATYYADSVKDRFTISRDDSKNSLYLQMNSLKTEDTAVYYCARWFRAMDYWGQ | |
| | GTLVTVSS | |
| 257 | EVQLVESGGGLVQPGGSLRLSCAASGFTFSMYGMHWVRQAPGKGLEWVSHIRSKS | Y01-14 VH |
| | SNYATYYADSVKDRFTISRDNAKNSLYLQMNSLRAEDTAVYYCARWFRAMDYWGQ | |
| | GTLVTVSS | |
| 258 | EVQLVESGGGLEQPGGSLRLSCAGSGFTFRMYGMHWVRQAPGKGLEWVSHIRSKS | 162-14 VH |
| | SNYATYYADSVKDRFTISRDNSKNTLYLQMNSLRAEDTAVYYCAKWFRAMDYWGQ | |
| | GTTVTVSS | |
| 259 | EVQLVESGGGLVQPGGSLKLSCAASGFTFSMYGMHWVRQASGKGLEWVGHIRSKS | VH73-14 VH |
| | SNYATYYADSVKDRFTISRDDSKNTAYLQMNSLKTEDTAVYYCTRWFRAMDYWGQ | |
| | GTTVTVSS | |
| 260 | DIVMTQSHKFMSTSVGDRVSITCKASQDVSTAVAWYQEKPGQSPKLLIYWASTRHT | Y0188-1 VL |
| | GVPDRFTGSGSGTDYTLTISSVQAEDLALYYCQQHYSTPLTFGAGTKLELK | |
| 261 | DIVVTQSPASLAVSLGQRATISCRASKSVSTSGYSYMHWYQQKPGQPPKLLIYLASNL | Y0188-2 VL |
| | ESGVPARFSGSGSGTDFTLNIHPVEEEDVAIYYCQHSRELPLTFGAGTKLELK | |
| 262 | DIQMTQSPSSLSASLGERVSLTCRASQEISGYLSWLQQKPDGTIKRLIYAASTLDSGVP | Y0188-3 VL |
| | KRFSGSRSGSDYSLTISSLESEDFADYYCLQYGSYPYTFGGGTKLEIK | |
| 263 | DIVLTQSPASLTVSLGQRATISCRASKSVSTSGYSYMHWYQQKPGQPPKLLIYLASNLE | Y0188-4 VL |
| | SGVPARFSGSGSGTDFTLNIHPVEEEDAATYYCQHSRELPITFGSGTKLEIK | |
| 264 | DIVLTQSPASLVVSLGQRATISCRASQSVSTSGYSYMHWYQQKPGQPPKLLIYLASNV | Y0188-6 VL |
| | QSGVPARFSGSGSGTDFTLNIHPVEEEDVATYYCHHNRDLPFTFGSGTKLEIK | |
| 265 | DIVVTQSPASLAVSLGQRATISCRASKSVSTSGYSYMHWYQQKPGQPPKLLIYLASNL | Y0188-8 VL |
| | ESGVPARFSGSGSGTDFTLNIHPVEEEDVAIYYCQHSRELPLTFGAGTKLELK | |
| 266 | DIVLTQSPASLAVSLGQRATISCRASKSVSASGYSYMHWYQQKPGQPPKLLIYLASNL | Y0188-9 VL |
| | QSGVPARFSGSGSGTDFTLNIHPVEEEDAATYYCQHSRELPPTFGGGTKLEIK | |
| 267 | DIVLTQSPASLAVFLGQRATISCRASKSVSTSGYSYMHWYQQKAGQPPKLLIYLASNL | Y0188-10 VL |
| | ESGVPARFSGSGSGTDFTLNIHPVEEEDAATYYCHHSRELPITFGSGTKLEMK | |
| 268 | DIVMTQSHKFMSTSVGDRVSITCKASQDVSTAVAWYQEKPGQSPKLLIYWASTRHT | Y0188-14 VL |
| | GVPDRFTGSGSGTDYTLTISSVQAEDLALYYCQQHYSTPLTFGAGTKLELK | |
| 269 | EIVLTQSPGTLSLSPGERATLSCKASQDVSTAVAWYQQKPGQAPRLLIYWASTRHTGI | Y01-14 VL |
| | PDRFSGSGSGTDFTLTISRLEPEDFAVYYCQQHYSTPLTFGQGTKVEIK | |
| 270 | DIVMTQSPLSLPVTPGEPASISCKASQDVSTAVAWYLQKSGQSPQLLIYWASTRHTG | 164-14 VL |
| | VPDRFSGSGSGTDFTLKISRVEAEDVGFYYCQQHYSTPLTFGQGTKLEIK | |
| 271 | DIVMTQSPDSLAVSLGERATINCKASQDVSTAVAWYQQKPGQPPKLLIYWASTRHT | KV4-14 VL |
| | GVPDRFSGSGSGTDFTLTISSLQAEDVAVYYCQQHYSTPLTFGGGTKVEIK | |
| 272 | DIQMTQSPSSLSASVGDRVTITCKASQDVSTAVAWYQQKPGKVPKLLIYWASTRHT | KV1-27-14 VL |
| | GVPSRFSGSGSGTDFTLTISSLQPEDVATYYCQQHYSTPLTFGGGTKVEIK | |
| 273 | DIQLTQSPSFLSASVGDRVTITCKASQDVSTAVAWYQQKPGKAPKLLIYWASTRHTG | KV1-9-14 VL |
| | VPSRFSGSGSGTEFTLTISSLQPEDFATYYCQQHYSTPLTFGGGTKVEIK | |
| 274 | DIQMTQSPSSLSASVGDRVTITCKASQDVSTAVAWYQQKPGKAPKLLLYWASTRHT | KV1-NL1-14 VL |
| | GVPSRFSGSGSGTDYTLTISSLQPEDFATYYCQQHYSTPLTFGGGTKVEIK | |
| 275 | AIRMTQSPFSLSASVGDRVTITCKASQDVSTAVAWYQQKPAKAPKLFIYWASTRHTG | KV1D-43-14 VL |
| | VPSRFSGSGSGTDYTLTISSLQPEDFATYYCQQHYSTPLTFGGGTKVEIK | |

What is claimed is:

1. A method for treating atopic dermatitis (AD) of the hand and/or foot, the method comprising:
selecting a subject having moderate-to-severe atopic dermatitis of the hand and/or foot, wherein the subject has a baseline hand and foot Investigator's Global Assessment (IGA) score ≥3, and wherein the subject has a history of inadequate response of atopic hand and/or foot dermatitis to topical medication or is a subject for whom topical treatment of atopic hand and/or foot dermatitis is medically inadvisable; and
administering to the subject one or more doses of an interleukin-4 receptor (IL-4R) antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

2. The method of claim 1, wherein the subject has a baseline hand and foot IGA score of 4.

3. The method of claim 1, wherein the subject is inadequately responsive to treatment with a topical corticosteroid (TCS) of medium or higher potency.

4. The method of claim 1, wherein the subject has a baseline hand and foot peak Pruritus Numerical Rating Score (NRS) ≥4.

5. The method of any one of claims 1 to 4, wherein the subject is an adult.

6. The method of claim 5, wherein the subject has had chronic atopic dermatitis of the hand and/or foot for at least 3 years.

7. The method of any one of claims 1 to 4, wherein the subject is an adolescent.

8. The method of claim 7, wherein the subject has had chronic atopic dermatitis of the hand and/or foot for at least one year.

9. The method of any one of claims 1 to 8, wherein the subject does not have irritant contact dermatitis or allergic contact dermatitis.

10. The method of any one of claims 1 to 9, wherein the subject does not have atopic dermatitis lesions on parts of the body other than the hands and/or feet.

11. The method of any one of claims 1 to 9, wherein the subject has mild atopic dermatitis of the body other than the hands and/or feet.

12. The method of any one of claims 1 to 9, wherein the subject has moderate-to-severe atopic dermatitis of the body other than the hands and/or feet.

13. The method of any one of claims 1 to 12, wherein the subject has a Modified Total Lesion Sign Score (mTLSS) for hand and foot 16.

14. The method of any one of claims 1 to 13, wherein the subject has a baseline hand and foot area of involvement of atopic dermatitis of at least 24%.

15. The method of any one of claims 1 to 14, wherein the subject has a Body Surface Area Involvement of Atopic Dermatitis (BSA) score <10% and/or an Eczema Area and Severity Index (EASI) score <21.

16. The method of any one of claim 1-11 or 13-15, wherein the subject:
(a) has a baseline whole-body IGA score ≤2; and/or
(b) has a baseline whole-body EASI score <16.

17. A method for treating atopic dermatitis (AD) of the hand and/or foot, the method comprising:
selecting a subject having moderate-to-severe AD of the hand and/or foot, wherein the subject is selected on the basis of having an mTLSS for hand and foot 16; and
administering to the subject one or more doses of an IL-4R antagonist, wherein the IL-4R antagonist is an anti-IL-4R antibody, or an antigen-binding fragment thereof, that comprises three heavy chain complementarity determining regions (HCDR1, HCDR2 and HCDR3) and three light chain complementarity determining regions (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence LGS, and the LCDR3 comprises the amino acid sequence of SEQ ID NO:8.

18. The method of claim 17, wherein the subject is further selected on the basis of having a baseline hand and foot IGA score ≥3, and/or a baseline hand and foot area of involvement of atopic dermatitis of at least 24%.

19. The method of claim 17 or 18, wherein the subject has a history of inadequate response of AD of the hand and/or foot to topical medication or is a subject for whom topical treatment of AD of the hand and/or foot is medically inadvisable.

20. The method of any one of claims 17 to 19, wherein the subject does not have atopic dermatitis lesions on parts of the body other than the hands and/or feet.

21. The method of any one of claims 17 to 19, wherein the subject has mild atopic dermatitis of the body other than the hands and/or feet.

22. The method of any one of claims 17 to 21, wherein the subject has:
(a) a baseline BSA score <10%;
(b) a baseline whole-body IGA score ≤2; and/or
(c) a baseline whole-body EASI score <16.

23. The method of any one of claims 17 to 22, wherein the subject is an adult.

24. The method of any one of claims 17 to 22, wherein the subject is an adolescent.

25. The method of any one of claims 17 to 24, wherein the subject is further selected on the basis of not having allergic contact dermatitis and/or not having irritant contact dermatitis.

26. The method of any one of claims 1 to 25, wherein the subject has chronic dry fissured hand and/or foot AD.

27. The method of any one of claims 1 to 25, wherein the subject has hyperkeratotic hand and/or foot AD.

28. The method of any one of claims 1 to 25, wherein the subject has dyshidrotic hand and/or foot AD.

29. The method of any one of claims 1 to 28, wherein the IL-4R antagonist is administered at a dose of about 50 mg to about 600 mg.

30. The method of any one of claims 1 to 29, wherein the IL-4R antagonist is administered as an initial dose of 100-600 mg followed by one or more subsequent doses of 50-300 mg.

31. The method of any one of claims 1 to 30, wherein the IL-4R antagonist is subcutaneously administered as an initial dose of 600 mg followed by one or more subsequent doses of 300 mg.

32. The method of any one of claims 1 to 30, wherein the IL-4R antagonist is subcutaneously administered as an initial dose of 300 mg followed by one or more subsequent doses of 300 mg.

33. The method of any one of claims 1 to 30, wherein the IL-4R antagonist is subcutaneously administered as an initial dose of 400 mg followed by one or more subsequent doses of 200 mg.

34. The method of any one of claims 1 to 30, wherein the IL-4R antagonist is subcutaneously administered as an initial dose of 200 mg followed by one or more subsequent doses of 200 mg.

35. The method of any one of claims 30 to 34, wherein each subsequent dose is administered one week or two weeks after the immediately preceding dose.

36. The method of any one of claim 1-4, 7-22, or 24-30, wherein the subject is an adolescent having a baseline weight 60 kg, and wherein the IL-4R antagonist is subcutaneously administered as an initial dose of 600 mg followed by one or more subsequent doses of 300 mg Q2W.

37. The method of any one of claim 1-4, 7-22, or 24-30, wherein the subject is an adolescent having a baseline weight <60 kg, and wherein the IL-4R antagonist is subcutaneously administered as an initial dose of 400 mg followed by one or more subsequent doses of 200 mg Q2W.

38. The method of any one of claim 1-6, 9-23, or 25-30, wherein the subject is an adult, and wherein the IL-4R antagonist is subcutaneously administered as an initial dose of 600 mg followed by one or more subsequent doses of 300 mg Q2W.

39. The method of any one of claims 1 to 38, wherein the IL-4R antagonist is administered for at least 16 weeks.

40. The method of any of claims 1 to 39, wherein the IL-4R antagonist is administered in combination with an emollient.

41. The method of any of claims 1 to 40, wherein the IL-4R antagonist is administered in combination with a topical AD medication.

42. The method of claim 41, wherein the topical AD medication is a topical corticosteroid (TCS).

43. The method of any one of claims 1 to 42, wherein treatment with the IL-4R antagonist results in the subject achieving an IGA hand and foot score of 0 or 1 by Week 16 after administration of a first dose of the IL-4R antagonist.

44. The method of any one of claims 1 to 43, wherein treatment with the IL-4R antagonist results in an improvement selected from the group consisting of:
a ≥4-point reduction in hand and foot peak Pruritis NRS, relative to a baseline hand and foot peak Pruritus NRS score for the subject, by Week 16 after administration of a first dose of the IL-4R antagonist;
a reduction in mTLSS for hand and foot score of at least 50%, relative to a baseline mTLSS for hand and foot score for the subject, by Week 16 after administration of a first dose of the IL-4R antagonist;
a reduction in hand and foot peak Pruritus NRS score of at least 50%, relative to a baseline hand and foot peak Pruritus NRS score for the subject, by Week 16 after administration of a first dose of the IL-4R antagonist;
a ≥4-point reduction in hand and foot peak Pain NRS, relative to a baseline hand and foot peak Pain NRS score for the subject, by Week 16 after administration of a first dose of the IL-4R antagonist;
a reduction in Hand Eczema Severity Index (HECSI) score of at least 50%, relative to a baseline HECSI score for the subject, by Week 16 after administration of a first dose of the IL-4R antagonist;
achievement of HECSI-75 by Week 16 after administration of a first dose of the IL-4R antagonist;
a reduction of at least 15% in the percent surface area of hand and foot involvement with AD, relative to a baseline percent surface area of hand and foot involvement with AD for the subject, by Week 16 after administration of a first dose of the IL-4R antagonist; and
a reduction of at least 40% in Quality of Life in Hand Eczema Questionnaire (QoLHEQ) score, relative to a baseline QoLHEQ score for the subject, by Week 16 after administration of a first dose of the IL-4R antagonist.

45. The method of any one of claims 1 to 44, wherein treatment with the IL-4R antagonist decreases the need for rescue treatment.

46. The method of any one of claims 1 to 45, wherein the anti-IL-4R antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:2.

47. The method of any one of claims 1 to 46, wherein the anti-IL-4R antibody comprises a heavy chain comprising the amino acid sequence of SEQ ID NO:9 and a light chain comprising the amino acid sequence of SEQ ID NO:10.

48. The method of any one of claims 1 to 47, wherein the IL-4R antagonist is dupilumab.

49. The method of any one of claims 1 to 48, wherein the IL-4R antagonist is contained in a container selected from the group consisting of a glass vial, a syringe, a pre-filled syringe, a pen delivery device, and an autoinjector.

50. The method of claim 49, wherein the IL-4R antagonist is contained in a pre-filled syringe.

51. The method of claim 50, wherein the pre-filled syringe is a single-dose pre-filled syringe.

52. The method of claim 49, wherein the IL-4R antagonist is contained in an autoinjector.

53. The method of claim 49, wherein the IL-4R antagonist is contained in a pen delivery device.
